SEQUENCE LISTING (6 sequences)

SEQ ID NO: 1 - DNA, 18 bp, mouse
caggagacgccggacagt

SEQ ID NO: 2 - DNA, 20 bp, mouse
cagggcaaagttgcactcaa

SEQ ID NO: 3 - DNA, 30 bp, mouse
aggaagatggcactgagatcgctattgtcc

SEQ ID NO: 4 - DNA, 20 bp, mouse
ctgccatcctcggaatcctt

SEQ ID NO: 5 - DNA, 22 bp, mouse
tggctcaaatcaaagtcctggt

SEQ ID NO: 6 - DNA, 28 bp, mouse
agggatcctcgccctgctgattctgatc

(19) United States
(12) Patent Application Publication
(10) Pub. No.: US 2009/0176656 A1
(43) Pub. Date: Jul. 9, 2009

(54) TISSUE REJECTION

(76) Inventor: Philip F. Halloran, Edmonton (CA)

Correspondence Address: FISH & RICHARDSON P.C., PO BOX 1022, MINNEAPOLIS, MN, 55440-1022, US

(21) Appl. No.: 12/374,639

(22) PCT Filed: Jul. 20, 2007
(86) PCT No.: PCT/CA2007/001295

§ 371 Date: Jan. 21, 2009

Related U.S. Application Data
(60) Provisional application No. 60/820,073, filed on Jul. 21, 2006.
(60) Provisional application No. 60/923,954, filed on Apr. 17, 2007.

(51) Int. Cl. C40B 30/04; C12Q 1/02; A01N 1/02; C12Q 1/68; C12M 1/00; C40B 40/08

(52) U.S. Cl. 506/9; 435/29; 435/1.1; 435/6; 435/287.1; 435/287.2; 506/17

(57) ABSTRACT

This document relates to methods and materials involved in detecting tissue injury and/or rejection (e.g., injury and/or rejection of transplanted tissue). For example, this document relates to methods and materials involved in the early detection of kidney tissue injury.

### TECHNICAL FIELD

This document relates to methods and materials involved in detecting tissue injury such as tissue injury that may occur with organ transplant rejection (alloimmune injury) or non-alloimmune injury. For example, this document relates to methods and materials involved in detecting tissue rejection.

### BACKGROUND

The transplantation of tissue from one human to another has been used for years to save lives and to improve the quality of lives. The first successful kidney transplant was performed in the mid-1950s between identical twin brothers. Since then, donors have grown to include close relatives, distant relatives, friends, and total strangers. In some cases, the recipient may reject the transplanted tissue. Thus, tissue rejection and tissue injury that may be due to alloimmune or non-alloimmune events is a concern for any recipient of transplanted tissue. If a clinician is able to recognize early signs of tissue rejection, anti-rejection drugs and other medication often can be used to reverse tissue rejection and manage injury. Further, understanding molecular mechanisms of injury and rejection will lead to development of improved diagnostics and therapeutics.

The success of organ transplantation is limited by the degree of injury resulting from the transplantation process (non-alloimmune injury), and by injury resulting from rejection (the alloimmune response). In kidney transplantation, the renal tubular epithelium is a key target of rejection. Changes in the epithelium have diagnostic significance in T cell mediated renal allograft rejection (TCMR). Entry of mononuclear inflammatory cells into the renal tubular epithelium during TCMR (Racusen et al. (1999) Kidney Int. 55:713-723) is associated with deterioration of renal function (Solez et al. (1993) Kidney Int. 43:1058-1067; and Solez et al. (1993) Kidney Int. 44:411-422). Tubulitis, associated with interstitial infiltration by mononuclear cells, is the principal lesion used to diagnose TCMR using the Banff schema (a pathology diagnostic system; Racusen et al. (supra). Kidneys also can be injured by antibody-mediated rejection (ABMR), the toxic effects of drugs, and through other mechanisms such as viral disease.

### SUMMARY

This document is based, in part, on the discovery of nucleic acids that are differentially expressed in tissue that is injured as compared to control tissue that is not injured. As such, this document relates to methods and materials involved in detecting tissue injury, such as injury inherent in an organ that is transplanted or is to be transplanted, or injury that occurs with organ transplantation (e.g., alloimmune injury associated with rejection, or non-alloimmune injury that can occur, for example, during surgery). For example, this document relates to methods and materials involved in early detection of tissue injury (e.g., tissue injury due to kidney rejection) and the assessment of a mammal's probability of rejecting tissue such as a transplanted organ. This document also relates to methods and materials involved in assessment of tissue quality and performance (e.g., assessment of donor organs for transplantation, prediction of whether an organ is at increased risk for developing delayed graft function (DGF) following transplantation, and assessment of transplanted organs and their potential to recover from alloimmune or non-alloimmune injury).

By analyzing the expression of nucleic acids as disclosed herein, tissue injury can be detected at a time point prior to the emergence of any visually-observable, histological sign of injury (e.g., in kidney tissue, tubulitis, loss of epithelial mass, marked reduction of E-cadherin and Ksp-cadherin, and redistribution to the apical membrane). In some embodiments, expression levels of “injury-and-repair induced transcripts” (IRITs), “not in isografts injury-and-repair induced transcripts” (NIRITs), “gamma-interferon suppressed transcripts” (GSTs), and “class I suppressed transcripts” (CISTs), including, for example, those listed in Tables 5-14, or expression levels of the solute carriers (Slcs) and renal transcripts (RTs) listed in Tables 1-4, can be assessed to determine whether or not tissue is injured, or to distinguish transplanted tissue that is injured from transplanted tissue that is not injured. In some embodiments, the expression level of gene profiles that significantly correlate with the sets referred to in Tables 1-14 (for example the gene set in Table 19 and/or the gene pathways in Table 21 that correlate with the gene profile shown in Tables 1 and 2, or the gene set in Table 20 and/or the gene pathways in Table 22 that correlate with the gene profile in Tables 7 and 8) can be assessed to determine whether or not tissue is injured, or to distinguish transplanted tissue that is injured from transplanted tissue that is not injured.

This document also relates to nucleic acid arrays that can be used to diagnose tissue injury in a mammal. Such arrays can, for example, allow clinicians to diagnose injury in a donor biopsy, diagnose tissue injury in a transplanted organ, or determine the potential for recovery of organ function in a transplanted organ, based on determination of the expression levels of nucleic acids that are differentially expressed in injured and/or rejected tissue as compared to control tissue that is not injured or rejected. The differential expression of such nucleic acids can be detected in injured tissue prior to the emergence of visually-observable, histological signs of tissue injury or rejection, allowing for early diagnosis of patients having injured transplanted tissue. Such diagnosis can help clinicians determine appropriate treatments for those patients. For example, a clinician who diagnoses a patient as having injured transplanted tissue can treat that patient with medication that suppresses tissue rejection and thus injury (e.g., immunosuppressants). In addition, better therapeutics can be developed that will treat or manage injury events.

In one aspect, this document features a method for detecting tissue injury, wherein the method comprises determining whether or not a tissue contains cells having an injury and repair profile, wherein the presence of the cells indicates that the tissue is injured. The mammal can be a human. The tissue can be from a biopsy. The tissue can be kidney tissue. The tissue can be tissue to be transplanted into a recipient. The tissue can be tissue that has been transplanted into a recipient. The determining step can comprise using PCR or a nucleic acid array, or can comprise using immunohistochemistry or an array for detecting polypeptides.

In another aspect, this document features a method for detecting tissue injury, wherein the method comprises determining whether or not a tissue contains cells having a not-in-isografts injury and repair profile, wherein the presence of the cells indicates that the tissue is injured. The mammal can be a human. The tissue can be from a biopsy. The tissue can be kidney tissue. The tissue can be tissue to be transplanted into a recipient. The tissue can be tissue that has been transplanted into a recipient. The determining step can comprise using PCR or a nucleic acid array, or can comprise using immunohistochemistry or an array for detecting polypeptides.

In another aspect, this document features a method for detecting tissue injury, wherein the method comprises determining whether or not a tissue contains cells having a gamma interferon (IFN-K) suppressed profile, wherein the presence of the cells indicates that the tissue is injured. The mammal can be a human. The tissue can be from a biopsy. The tissue can be kidney tissue. The tissue can be tissue to be transplanted into a recipient. The tissue can be tissue that has been transplanted into a recipient. The determining step can comprise using PCR or a nucleic acid array, or can comprise using immunohistochemistry or an array for detecting polypeptides.

In another aspect, this document features a method for detecting tissue injury, wherein the method comprises determining whether or not a tissue contains cells having a class I suppressed profile, wherein the presence of the cells indicates that the tissue is injured. The mammal can be a human. The tissue can be from a biopsy. The tissue can be kidney tissue. The tissue can be tissue to be transplanted into a recipient. The tissue can be tissue that has been transplanted into a recipient. The determining step can comprise using PCR or a nucleic acid array, or can comprise using immunohistochemistry or an array for detecting polypeptides.

In yet another aspect, this document features a method for detecting tissue injury, wherein the method comprises determining whether or not a tissue contains cells having a renal transcript (RT) profile, wherein the presence of the cells indicates that the tissue is injured. The mammal can be a human. The tissue can be from a biopsy. The tissue can be kidney tissue. The tissue can be tissue to be transplanted into a recipient. The tissue can be tissue that has been transplanted into a recipient. The determining step can comprise using PCR or a nucleic acid array, or can comprise using immunohistochemistry or an array for detecting polypeptides.

In another aspect, this document features a method for detecting tissue injury, wherein the method comprises determining whether or not a tissue contains cells having a solute carrier (Slc) profile, wherein the presence of the cells indicates that the tissue is injured. The mammal can be a human. The tissue can be from a biopsy. The tissue can be kidney tissue. The tissue can be tissue to be transplanted into a recipient. The tissue can be tissue that has been transplanted into a recipient. The determining step can comprise using PCR or a nucleic acid array, or can comprise using immunohistochemistry or an array for detecting polypeptides.

This document also features a method for assessing whether a tissue is at risk for delayed graft function (DGF), wherein the method comprises determining whether or not a tissue contains cells having an injury and repair profile, a non-in-isografts injury and repair profile, an IFN-K suppressed profile, a class I suppressed profile, a RT profile, or a Slc profile, wherein the presence of the cells indicates that the tissue is at risk for DGF. The mammal can be a human. The tissue can be from a biopsy. The tissue can be kidney tissue. The tissue can be tissue to be transplanted into a recipient. The tissue can be tissue that has been transplanted into a recipient. The determining step can comprise using PCR or a nucleic acid array, or can comprise using immunohistochemistry or an array for detecting polypeptides.

In another aspect, this document features a method for predicting whether a transplanted tissue will recover from injury, wherein the method comprises determining whether or not a tissue contains cells having an injury and repair profile, a non-in-isografts injury and repair profile, an IFN-K suppressed profile, a class I suppressed profile, a RT profile, or a Slc profile, wherein the presence of the cells indicates that the tissue is not likely to recover from injury. The mammal can be a human. The tissue can be from a biopsy. The tissue can be kidney tissue. The tissue can be tissue to be transplanted into a recipient. The tissue can be tissue that has been transplanted into a recipient. The determining step can comprise using PCR or a nucleic acid array, or can comprise using immunohistochemistry or an array for detecting polypeptides.

In still another aspect, this document features a method for detecting tissue injury, wherein the method comprises determining whether or not a tissue contains cells having an injury and repair correlated profile or an Slc correlated profile, wherein the presence of the cells indicates that the tissue is injured. The mammal can be a human. The tissue can be from a biopsy. The tissue can be kidney tissue. The tissue can be tissue to be transplanted into a recipient. The tissue can be tissue that has been transplanted into a recipient. The determining step can comprise using PCR or a nucleic acid array, or can comprise using immunohistochemistry or an array for detecting polypeptides.

This document also features a method for detecting tissue injury, comprising determining whether or not a tissue contains cells having increased activity of biochemical pathways that correlate with an injury and repair profile, with an Slc profile, with a non-in-isografts injury and repair profile, with a gamma interferon suppressed profile, with a class I suppressed profile, or with an RT profile, wherein the presence of the cells indicates that the tissue is injured.

In another aspect, this document features a nucleic acid array comprising at least 20 nucleic acid molecules, wherein each of the at least 20 nucleic acid molecules has a different nucleic acid sequence, and wherein at least 50 percent of the nucleic acid molecules of the array comprise a sequence from nucleic acid selected from the group consisting of the nucleic acids listed in Tables 1-14, 19, and 20. The array can comprise at least 50 nucleic acid molecules, wherein each of the at least 50 nucleic acid molecules has a different nucleic acid sequence. The array can comprise at least 100 nucleic acid molecules, wherein each of the at least 100 nucleic acid molecules has a different nucleic acid sequence. Each of the nucleic acid molecules that comprise a sequence from nucleic acid selected from the group can comprise no more than three mismatches. At least 75 percent of the nucleic acid molecules of the array can comprise a sequence from nucleic acid selected from the group. At least 95 percent of the nucleic acid molecules of the array can comprise a sequence from nucleic acid selected from the group. The array can comprise glass. The at least 20 nucleic acid molecules can comprise a sequence present in a human.

In another aspect, this document features a computer-readable storage medium having instructions stored thereon for causing a programmable processor to determine whether one or more nucleic acids listed in Tables 5-14, and the third column of Table 20 are present in a tissue sample at elevated levels. The computer-readable storage medium can further comprise instructions stored thereon for causing a programmable processor to determine whether one or more of the nucleic acids listed in Tables 5-14, and the third column of 20 is expressed at a greater level in the tissue sample than in a control tissue sample.

In another aspect, this document features a computer-readable storage medium having instructions stored thereon for causing a programmable processor to determine whether one or more nucleic acids listed in Tables 1-4 and the third column of Table 19 are present in a tissue sample at decreased levels. The computer-readable storage medium can further comprise instructions stored thereon for causing a programmable processor to determine whether one or more of the nucleic acids listed in Tables 1-4 and the third column of Table 19 is expressed at a lower level in the tissue sample than in a control tissue sample.

In yet another aspect, this document features an apparatus for determining whether a tissue is injured, the apparatus comprising: one or more collectors for obtaining signals representative of the presence of one or more nucleic acids listed in Tables 1-14, 19, and 20 in a sample from the tissue; and a processor for analyzing the signals and determining whether the tissue is injured. The one or more collectors can be configured to obtain further signals representative of the presence of the one or more nucleic acids in a control sample.

In another aspect, this document features a method for detecting tissue rejection. The method comprises, or consists essentially of, determining whether or not tissue transplanted into a mammal contains cells that express a reduced level of a cadherin polypeptide or a transporter polypeptide, wherein the presence of the cells indicates that the tissue is being rejected. The mammal can be a human. The tissue can be kidney tissue. The tissue can be a kidney. The method can comprise determining whether or not the tissue contains cells that express a reduced level of the cadherin polypeptide. The cadherin polypeptide can be an E-cadherin polypeptide or a Ksp-cadherin polypeptide. The method can comprise determining whether or not the tissue contains cells that express a reduced level of the transporter polypeptide. The transporter polypeptide can be selected from the group consisting of Slc2a2, Slc2a4, Slc2a5 Slc5a1, Slc5a2, Slc5a10, Slc7a7, Slc7a8, Slc7a9, Slc7a10, Slc7a12, Slc7a13, Slc1a4, Slc3a1, Slc1a1, aquaporin 1, aquaporin 2, aquaporin 3, aquaporin 4, ABC transporter (e.g., a member of the ABC transporter polypeptide family), solute carrier, and ATPase polypeptides. The determining step can comprise measuring the level of mRNA encoding the cadherin polypeptide or the transporter polypeptide. The determining step can comprise measuring the level of the cadherin polypeptide or the transporter polypeptide. The method can comprise determining whether or not the tissue contains cells that express the cadherin polypeptide or the transporter polypeptide at a level less than the average level of expression exhibited in cells from control tissue that has not been transplanted. The determining step can comprise determining whether or not a sample contains the cells, wherein the sample comprises cells, was obtained from tissue that was transplanted into the mammal, and was obtained from the tissue within fifteen days of the tissue being transplanted into the mammal.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description and drawings below. Other features, objects, and advantages of the invention will be apparent from the description and the drawings, and from the claims.

### BRIEF DESCRIPTION OF THE DRAWINGS

FIG. 1 is a depiction of the algorithm used to develop the unique IRIT list.

FIG. 2 is a dendrogram for donor (implant) biopsies of 42 deceased donor (DD) and 45 living donor (LD) kidneys. The DIANA dendrogram is based on all 7376 inter-quartile range- (IQR-) filtered probesets. Black boxes indicate pairs, and arrows indicate delayed graft function (DGF).

FIG. 3 is a graph showing principal component analysis (PCA) of the transcriptome of 87 donor (implant) biopsies, based on the same set of 7376 IQR-filtered probesets as clustered in FIG. 2. L, samples from 45 living donors; D, samples from 42 deceased donors; boxes, kidneys experiencing post-transplant delayed graft function; green, samples in cluster 1; orange, samples in cluster 2; black, samples in cluster 3 as shown in FIG. 2.

FIG. 4 is a chart showing pathogenesis based transcript (PBT) scores calculated for the 3 clusters shown in FIG. 2. Only those probesets passing the non-specific (IQR) filtering step were used to calculate the scores. Cluster 3 (“high-risk”) is subdivided into samples with (n=8) and without (n=13) DGF. LD, living donor implants, Cluster 1; low risk, Cluster 2. PBT scores are defined as fold-change relative to the nephrectomy controls, averaged over all probesets within each PBT.

FIG. 5 is a chart showing p-values from Bayesian t-tests comparing inter-cluster PBT scores. p-values have been corrected using Benjamini and Hochberg's false discovery rate method. The Cluster 3 (“high-risk”) group has been subdivided into samples with and without DGF.

FIG. 6 is a graph plotting ROC curves for Principal Component 1 (PC1), showing PCA 1's value in predicting DGF status in the 42 DD kidneys. PC1 was based on all probesets passing the IQR-filter, and on all 87 (LD+DD) samples. Solid line, the smoothed-average ROC curve of all 42 leave-one-out cross validated (LOOCV) estimates; horizontal bars, medians in boxplots; dotted lines, non-smoothed individual ROC curves for each of the LOOCV estimates.

FIG. 7 is a graph plotting ROC curves showing individual PBT scores (RTs, tGRITs, and mCATs) and PC1 scores in predicting DGF status in the 42 DD kidneys. The PC1 scores were based on genes that were both IQR filtered and PBTs. Horizontal bars, medians in boxplots; dotted lines, non-smoothed individual ROC curves for each of the LOOCV estimates.

FIG. 8. is a table showing the correlation of gene sets with function (GFR) at the time of biopsy and 3 months after biopsy.

FIG. 9. is a table showing the correlation of gene sets with the degree of loss of function/GFR before biopsy (all gene sets; center column) and recovery of function/GFR after biopsy (IRITs, GSTs, CISTs; right column).

FIG. 10 is a table showing that the best correlations between renal function (GFR) and gene sets are with the IRITs, particularly with IRITsD3 and IRITsD5.

FIG. 11 is a table showing that the best correlations between degree of loss of function/GFR and gene sets are with the IRITs, especially the IRITsD3 and IRITsD5.

FIG. 12: Histology of rejecting kidneys (CBA into B6 transplants; PAS staining). A) Day 5 transplant with periarterial infiltration (magnification 20×). B) Day 5 transplant showing no tubulitis (magnification 100×). C) Day 7 allograft showing interstitial infiltration (magnification 20×). D) Day 7 transplant with mild tubulitis (magnification 100×). E) Day 21 allograft showing interstitial infiltration and edema (magnification 20×). F) Day 21 transplant with marked tubulitis (arrows) and distorted tubules (magnification 100×).

FIG. 13: Real time RT-PCR analysis of CD103 mRNA expression. A) normal kidney (NCBA) and allografts (CBA into B6) at days 5, 7, and 21 post transplant. B) NCBA, contralateral CD103^{-/-} host kidneys, CBA kidneys rejecting in CD103^{-/-} or in wild-type Balb/c hosts at day 21 post transplant. Values are fold changes relative to control kidney (NCBA), expressed as mean±SE. Assays were done in duplicate.

FIG. 14: Histology of allografts rejecting in wild-type (CBA into Balb/c) or CD103^{-/-} (CBA into CD103^{-/-}) hosts at day 21 post transplant. A) Allograft in wild-type host showing interstitial edema, marked tubulitis (arrows) and distorted tubules (PAS staining, magnification 60×). B) Allograft in CD103−/− host showing interstitial edema, marked tubulitis (arrows) and distorted tubules, indistinguishable from wild-type (PAS staining, magnification 60×). C). Electron microscopy of tubulitis lesions in allografts rejecting in wild-type hosts D) Electron microscopy of tubulitis lesions in allografts rejecting in CD103^{-/-} hosts. (Lymphocytes within the tubular epithelial cells; lymphocytes in the interstitium; tubular basement membrane).

FIG. 15: Expression of epithelial transporter transcripts (glucose transporters, amino acid transporters, aquaporins) in isografts and rejecting allografts (CBA into B6) at days 5, 7, and 21 post-transplant, determined by Affymetrix microarrays MOE 430A.

FIG. 16: E-cadherin and Ksp-cadherin in rejecting allografts. A) Real time RT-PCR analysis of mRNA expression of cadherins in rejecting kidney (CBA into B6). Values are fold changes relative to control (CBA) kidney, expressed as means±SE (n=2, three kidneys in each pool). Assays were done in duplicate. B) Western blot analysis of E-cadherin and Ksp-cadherin expression. Fold changes were calculated from the band intensity ratio of Tx (transplant:CBA into B6) versus C (contralateral kidney: B6). Shown are means±SE, n=3. Basal levels of cadherins did not differ significantly between normal (CBA mice) and contralateral kidneys (B6 mice). C) E-cadherin and Ksp-cadherin mRNA expression in allografts rejecting in wild-type Balb/c (WT) or CD103^{-/-} hosts at day 21 post transplant.

FIG. 17: Immunohistochemical staining of E-cadherin and Ksp-cadherin (magnification 100×). Arrows show localization of cadherins. At day 7 post transplant, E-cadherin was localized to the basolateral membrane A) in B6 host kidney and B) in rejecting allografts (CBA into B6). At day 21 post transplant, E-cadherin staining was decreased with some redistribution to the apical membrane C) in allografts rejecting in wild-type hosts (CBA into B6) and D) in allografts rejecting in CD103^{-/-} hosts (CBA into CD103^{-/-}). E) Ksp-cadherin was localized to the basolateral membrane in normal CBA kidney (control). Ksp-cadherin was decreased in rejecting allografts F) in wild-type hosts (CBA into B6) at day 7 post transplant, G) in wild-type hosts (CBA into B6) at day 21 post transplant and H) in CD103^{-/-} hosts (CBA into CD103^{-/-}) at day 21 post transplant.

### DETAILED DESCRIPTION

This document provides methods and materials involved in detecting tissue injury (e.g., injury inherent in a tissue to be transplanted, or tissue injury that may occur with organ transplantation, including alloimmune and non-alloimmune injury) and assessing the potential for recovery of organ function. For example, this document provides methods and materials that can be used to determine whether a tissue is injured or susceptible to injury and delay in function. In some cases, a mammal can be diagnosed as having transplanted tissue that is injured (due to rejection or not) or likely to be injured if it is determined that the tissue contains cells that express altered levels of one or more nucleic acid transcripts, as described herein.

As described herein, the expression levels of particular transcripts, including mouse and human “injury-and-repair induced transcripts” (IRITs), “not in isografts injury-and-repair induced transcripts” (NIRITs), “gamma-interferon suppressed transcripts” (GSTs), and “class I suppressed transcripts” (CISTs) can be used to distinguish tissue (e.g., transplanted tissue) that is injured from tissue that is not injured. This document also is based, in part, on the discovery that the expression levels of mouse “cytotoxic T lymphocyte-associated transcripts” (CATs) and “true gamma-interferon dependent and rejection-induced transcripts” (tGRITs) can be used to distinguish tissue (e.g., transplanted tissue) that is being rejected from tissue that is not being rejected as disclosed, for example, in U.S. Publication Nos. 2006/0269948 and 2006/0269949. For example, the expression levels of nucleic acids listed in Tables 5-14 can be assessed in transplanted tissue to determine whether or not that transplanted tissue is injured. In addition, the description provided herein is based, in part, on the discovery that the expression levels of renal transcripts (RTs) such as those listed in Tables 3 and 4, including the solute carriers (Slcs) listed in Tables 1 and 2, can be used to distinguish tissue that is injured (e.g., transplanted tissue that is injured) from uninjured tissue. In addition, gene lists and pathways have been identified that are significantly positively or negatively correlated with the gene profiles described in Tables 1-14 (e.g., the gene set in Table 19 and/or the gene pathways in Table 21 that correlate with the gene profile shown in Tables 1 and 2, or the gene set in Table 20 and/or the gene pathways in Table 22 that correlate with the gene profile in Tables 7 and 8). These gene sets and pathways can be used to distinguish tissue that is injured from tissue that is not injured.

For example, a mammal can be diagnosed as having transplanted tissue that is injured if it is determined that the tissue contains cells expressing elevated levels of one or more IRITs, NIRITs, GSTs, and/or CISTs, or that express elevated levels one or more of the nucleic acids listed in Tables 5-14. In some embodiments, a mammal can be diagnosed as having transplanted tissue that is injured if it is determined that the tissue contains cells that express reduced levels of one or more Slcs and RTs listed in Tables 1-4. In some cases, a mammal can be diagnosed as having transplanted tissue that is injured if it is determined that the tissue contains cells that express gene lists and/or pathways that are significantly positively or negatively correlated with the gene profiles described in Tables 1-14 (e.g., the gene set in Table 19 and/or the gene pathways in Table 21 that correlate with the gene profile shown in Tables 1 and 2, or the gene set in Table 20 and/or the gene pathways in Table 22 that correlate with the gene profile in Tables 7 and 8).

The term “injury and repair-induced transcripts” or “IRITs” refers to transcripts that are increased in isografts at least once between day 1 and day 21, as compared to normal kidney, excluding allogeneic effects as well as T cell-associated, macrophage associated, and IFN-γ inducible transcripts. Thus, IRITs indicate non-alloimmune effects, such as injury caused by surgery or ischemia reperfusion, for example. The ATN model discussed herein demonstrates ischemia reperfusion injury. In some embodiments, an “IRIT” is identified based on expression that is at least two-fold in kidney isografts as compared to normal kidney. Examples of IRITs include, without limitation, the nucleic acids listed in Tables 7-10. Some IRITs, such as those listed in Table 9, also are primary macrophage associated transcripts (MATs). These transcripts indicate non-alloimmune injury involving innate immune responses.

Some gene sets and pathways have been found to be positively or negatively correlated with IRITs. For example, the genes listed in the first column of Table 20 are negatively correlated with IRITs, while the genes listed in the third column of Table 20 are positively correlated with IRITs. Further, the pathways listed in the left column of Table 22 are negatively correlated with IRITs, while the pathways listed in the right column of Table 22 are positively correlated with IRITs. Thus, increased expression of the positively correlated genes listed in Table 20, increased activity of the positively correlated pathways listed in Table 22, decreased expression of the negatively correlated genes listed in Table 20, or decreased activity of the negatively correlated pathways listed in Table 22, can indicate tissue injury (e.g., non-alloimmune injury).

The term “(not in isografts) injury and repair induced transcripts” or “NIRITs” as used herein refers to transcripts that are elevated in kidney allografts vs. isografts at least once between day 1 and day 42 post transplant in WT hosts, excluding transcriptomes of infiltrating T cells, B cells and macrophages, IFN-K inducible genes, cytotoxic T cell associated transcripts, IFN-γ dependent rejection induced transcripts, and transcripts showing strain differences. A “NIRIT” can be identified based on expression that is increased in kidney allografts as compared to control kidneys, but not increased in kidney isografts as compared to control kidneys. Thus, NIRITs indicate injury that occurs in the parenchyma of the kidney (i.e., the transcriptome of the infiltrating cell compartments have been “removed”) and is due to an alloimmune response rather than a non-alloimmune response. Examples of NIRITs include, without limitation, the nucleic acids listed in Tables 5 and 6.

Some nucleic acids that are differentially expressed in tissue that is injured as compared to control tissue that is not injured can be nucleic acids that are suppressed by gamma interferon (IFN-γ). The term “IFN-γ suppressed transcripts” or “GSTs” as used herein refers to transcripts that are expressed in IFN-γ receptor deficient kidney allograft tissue at a level that is greater than the level of expression in WT kidney allograft tissue. In some embodiments, for example, a “GST” is identified based on expression that is increased at least two-fold in IFN-γ receptor deficient kidney allograft tissue as compared to the level of expression in WT kidney allograft tissue. GSTs indicate the underlying alternative inflammatory response to alloimmune and non-alloimmune injury. Examples of GSTs include, without limitation, the nucleic acids listed in Tables 11 and 12.

Some nucleic acids can be suppressed by class-I proteins (e.g., MHC class Ia and/or Ib proteins such as the Tap1 transporter and beta 2 microglobulin). The term “class I suppressed transcripts” or “CISTs” as used herein refers to transcripts that are expressed in class I protein (e.g., Tap1 transporter and beta 2 microglobulin) deficient kidney allograft tissue at a level that is greater than the expression in WT kidney allograft tissue. In some embodiments, for example, a “CIST” is identified based on expression that is increased at least two-fold in class I deficient kidney allograft tissue as compared to the level of expression in WT kidney allograft tissue. CISTs indicate the underlying alternative inflammatory response that occurs to alloimmune and non-alloimmune injury, and demonstrates the involvement of IFN-K in the process. Examples of CISTs include, without limitation, the nucleic acids listed in Tables 13 and 14.

In some embodiments, a nucleic acid can be included in two or more of the categories described herein. For example, some nucleic acids can be considered to be GSTs and CISTs. Elevated levels of such GST/CIST nucleic acids can indicate injury in allograft transplants, for example.

The RTs listed in Tables 3 and 4 are renal transcripts that are reduced in allografts and isografts with injury. These transcripts reflect non-alloimmune injury due, for example, to surgical stress, ischemia reperfusion, and other causes, as well as ongoing additional injury effects that occur in alloimmune rejection. The Slcs listed in Tables 1 and 2 are renal solute carrier transcripts that are decreased in allografts and isografts with injury. Like the RTs, the Slcs reflect non-alloimmune injury and alloimmune injury.

Some gene sets and pathways have been found to be positively or negatively correlated with Slcs. For example, the genes listed in the first column of Table 19 are negatively correlated with Slcs, while the genes listed in the third column of Table 19 are positively correlated with Slcs. Further, the pathways listed in the left column of Table 21 are negatively correlated with Slcs, while the pathways listed in the right column of Table 21 are positively correlated with Slcs. Thus, reduced expression of the positively correlated genes listed in Table 19, reduced activity of the positively correlated pathways listed in Table 21, increased expression of the negatively correlated genes listed in Table 19, or increased activity of the negatively correlated pathways listed in Table 21 can indicate tissue injury (e.g., non-alloimmune injury or alloimmune injury).

Some nucleic acids can be expressed in T lymphocytes. The term “cytotoxic T lymphocyte-associated transcripts” or “CATs” refers to transcripts that are not usually expressed in kidney but are induced in rejection, and that may reflect T cells recruited to the graft. Examples of CATs include, without limitation, the nucleic acids listed in Table 15. These transcripts are diagnostic for allograft rejection and are referred to in co-pending U.S. Publication No. 2006/0269948.

Some nucleic acids can be regulated by IFN-γ and induced by rejection. The term “true interferon gamma dependent and rejection-induced transcripts” or “tGRITs” refers to rejection-induced transcripts that are IFN-γ-dependent in rejection, and also are unique transcripts that are increased at least 2-fold by rIFN-γ. See, co-pending U.S. Publication No. 2006/0269949. Examples of tGRITs include, without limitation, the nucleic acids listed in Table 16, which can be diagnostic for allograft rejection.

The term “transcript” as used herein refers to an mRNA identified by one or more numbered Affymetrix probe sets, while a “unique transcript” is an mRNA identified by only one probe set.

In some embodiments, a tissue can be identified as being injured if it is determined that the tissue contains cells having an injury and repair profile, a not-in-isografts injury and repair profile, an IFN-K suppressed profile, or a class I suppressed profile. As used herein, the term “injury and repair profile” refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, 25, 26, 27, or more) of the nucleic acids or polypeptides encoded by the nucleic acids listed in Tables 7-10 is present at an elevated level.

The term “not-in-isografts injury and repair profile,” as used herein, refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, 25, 26, 27, or more) of the nucleic acids or polypeptides encoded by the nucleic acids listed in Tables 5 and 6 is present at an elevated level.

As used herein, the term “IFN-K suppressed profile” refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, 25, 26, 27, or more) of the nucleic acids or polypeptides encoded by the nucleic acids listed in Tables 11 and 12 is present at an elevated level.

The term “class I suppressed profile” refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, 25, 26, 27, or more) of the nucleic acids or polypeptides encoded by the nucleic acids listed in Tables 13 and 14 is present at an elevated level.

In some embodiments, a tissue can be identified as being injured if it is determined that the tissue contains cells having a RT profile or a Slc profile. As used herein, the term “RT profile” refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, 25, 26, 27, or more) of the nucleic acids or polypeptides encoded by the nucleic acids listed in Tables 3 and 4 is present at a reduced level, and the term “Slc profile” refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, 25, 26, 27, or more) of the nucleic acids or polypeptides encoded by the nucleic acids listed in Tables 1 and 2 is present at an reduced level.

In some embodiments, a tissue can be identified as being injured if it is determined that the tissue contains cells having a quantitative injury and repair profile, a quantitative not-in-isografts injury and repair profile, a quantitative IFN-K suppressed profile, or a quantitative class I suppressed profile. As used herein, the term “quantitative injury and repair profile” refers to a nucleic acid or polypeptide profile in a sample where one tenth or more of the nucleic acids or polypeptides encoded by the nucleic acids listed in Tables 7-10 are present at an elevated level. For example, a quantitative human injury and repair profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 8 are present at an elevated level.

The term “quantitative not-in-isografts injury and repair profile” as used herein refers to a nucleic acid or polypeptide profile in a sample where one tenth or more of the nucleic acids or polypeptides encoded by the nucleic acids listed in Tables 5 and 6 are present at an elevated level. For example, a human not-in-isografts injury and repair profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 6 are present at an elevated level.

The term “quantitative IFN-K suppressed profile” as used herein refers to a nucleic acid or polypeptide profile in a sample where one tenth or more of the nucleic acids or polypeptides encoded by the nucleic acids listed in Tables 11 and 12 are present at an elevated level. For example, a human IFN-K suppressed profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 12 are present at an elevated level.

The term “quantitative class I suppressed profile” as used herein refers to a nucleic acid or polypeptide profile in a sample where one tenth or more of the nucleic acids or polypeptides encoded by the nucleic acids listed in Tables 13 and 14 are present at an elevated level. For example, a human class I suppressed profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 14 are present at an elevated level.

In some embodiments, a tissue can be identified as being injured if it is determined that the tissue contains cells having a quantitative RT profile, or a quantitative Slc profile. The term “quantitative RT profile” as used herein refers to a nucleic acid or polypeptide profile in a sample where one tenth or more of the nucleic acids or polypeptides encoded by the nucleic acids listed in Tables 3 and 4 are present at a reduced level. For example, a quantitative human RT profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 4 are present at a reduced level.

The term “quantitative Slc profile” as used herein refers to a nucleic acid or polypeptide profile in a sample where one tenth or more of the nucleic acids or polypeptides encoded by the nucleic acids listed in Tables 1 and 2 are present at a reduced level. For example, a quantitative human Slc profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 2 are present at a reduced level.

In some embodiments, a tissue can be identified as being injured if it is determined that the tissue contains cells having an injury and repair positively correlated profile or an injury and repair negatively correlated profile. As used herein, the term “injury and repair positively correlated profile” refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, or 25) of the nucleic acids or polypeptides encoded by the nucleic acids listed in column 3 of Table 20 is present at an elevated level. The term “injury and repair negatively correlated profile” refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, or 25) of the nucleic acids or polypeptides encoded by the nucleic acids listed in column 1 of Table 20 is present at an elevated level. The presence of an injury and repair positively correlated profile can indicate that a tissue is injured. The presence of an injury and repair negatively correlated profile also can indicate that a tissue is injured.

In some embodiments, a tissue can be identified as being injured if it is determined that the tissue contains cells having a quantitative injury and repair positively correlated profile or a quantitative injury and repair negatively correlated profile. The term “quantitative injury and repair positively correlated profile” as used herein refers to a nucleic acid or polypeptide profile in a sample where one tenth or more of the nucleic acids or polypeptides encoded by the nucleic acids listed in the third column of Table 20 are present at an elevated level. For example, a quantitative injury and repair positively correlated profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in the third column of Table 20 are present at an elevated level. The term “quantitative injury and repair negatively correlated profile” as used herein refers to a nucleic acid or polypeptide profile in a sample where one tenth or more of the nucleic acids or polypeptides encoded by the nucleic acids listed in the first column of Table 20 are present at an elevated level. For example, a quantitative injury and repair negatively correlated profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in the first column of Table 20 are present at an elevated level.

In some embodiments, a tissue can be identified as being injured if it is determined that the tissue contains cells having an Slc positively correlated profile or an Slc negatively correlated profile. As used herein, the term “Slc positively correlated profile” refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, or 25) of the nucleic acids or polypeptides encoded by the nucleic acids listed in the third column of Table 19 is present at a reduced level. The term “Slc negatively correlated profile” refers to a nucleic acid or polypeptide profile in a sample (e.g., a sample of tissue that is transplanted or is to be transplanted) in which one or more than one (e.g., 1, 2, 3, 4, 5, 6, 7, 8, 9, 10, 11, 12, 13, 14, 15, 16, 17, 18, 19, 20, 21, 22, 23, 24, or 25) of the nucleic acids or polypeptides encoded by the nucleic acids listed in the first column of Table 19 is present at a reduced level. The presence of an Slc positively correlated profile can indicate that a tissue is injured. The presence of an Slc negatively correlated profile also can indicate that a tissue is injured.

In some embodiments, a tissue can be identified as being injured if it is determined that the tissue contains cells having a quantitative Slc positively correlated profile or a quantitative Slc negatively correlated profile. The term “quantitative Slc positively correlated profile” as used herein refers to a nucleic acid or polypeptide profile in a sample where one tenth or more of the nucleic acids or polypeptides encoded by the nucleic acids listed in the third column of Table 19 are present at a reduced level. For example, a quantitative Slc positively correlated profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in the third column of Table 19 are present at a reduced level. The term “quantitative Slc negatively correlated profile” as used herein refers to a nucleic acid or polypeptide profile in a sample where one tenth or more of the nucleic acids or polypeptides encoded by the nucleic acids listed in the first column of Table 19 are present at a reduced level. For example, a quantitative Slc negatively correlated profile can be a nucleic acid or polypeptide profile in a sample where 10%, 12%, 15%, 18%, 20%, 22%, 23%, 25%, 27%, 29%, 30%, 32%, 33%, 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in the first column of Table 19 are present at a reduced level.

The methods and materials provided herein can be used to detect tissue injury (e.g., tissue rejection) in any mammal, including, without limitation, a human, monkey, horse, dog, cat, cow, pig, mouse, or rat. In addition, the methods and materials provided herein can be used to detect injury of any type of tissue including, without limitation, kidney, heart, liver, pancreas, and lung tissue. For example, the methods and materials provided herein can be used to determine whether or not a human who received a kidney transplant is experiencing injury of the transplanted kidney.

Any type of sample containing cells can be used to determine whether or not transplanted tissue, tissue that is not transplanted, or tissue that is to be transplanted (e.g., donor biopsy) contains cells that express one or more IRITs, NIRITs, GSTs, and or CISTs, or that express one or more of the nucleic acids or polypeptides encoded by the nucleic acids listed in Tables 5-14, at elevated levels. Similarly, any type of sample containing cells can be used to determine whether or not transplanted tissue, tissue that is not transplanted, or tissue that is to be transplanted (e.g., donor biopsy) contains cells that express one or more of the nucleic acids or polypeptides encoded by the nucleic acids listed in Tables 1-4 at decreased levels. Further, any type of sample containing cells can be used to determine whether transplanted tissue, tissue that is not transplanted, or tissue that is to be transplanted (e.g., donor biopsy) contains cells that express one or more nucleic acids that significantly positively or negatively correlate with nucleic acids listed in Tables 1-14. For example, biopsy (e.g., punch biopsy, aspiration biopsy, excision biopsy, needle biopsy, or shave biopsy), tissue section, lymph fluid, blood, and synovial fluid samples can be used. In some embodiments, a tissue biopsy sample can be obtained directly from a tissue that has been transplanted or is to be transplanted. In some embodiments, a lymph fluid sample can be obtained from one or more lymph vessels that drain from the tissue. A sample can contain any type of cell including, without limitation, cytotoxic T lymphocytes, CD4⁺ T cells, B cells, peripheral blood mononuclear cells, macrophages, kidney cells, lymph node cells, or endothelial cells.

Additional examples of Slcs, RTs, IRITs, NIRITs, GSTs, and CISTs, as well as other transcripts with altered expression levels in injured tissues (e.g., genes in pathways related to glutathione metabolism, fatty acid elongation, and cell communication) can be identified using the procedures described herein. For example, the procedures described in Examples 1 and 2 can be used to identify RTs other than those listed in Tables 1-4, the procedures described in Examples 1 and 4 can be used to identify IRITs other than those listed in Tables 7-10, the procedures described in Examples 1 and 3 can be used to identify NIRITs other than those listed in Tables 5 and 6, the procedures described in Examples 1 and 5 can be used to identify GSTs other than those listed in Tables 11 and 12, and the procedures described in Examples 1 and 6 can be used to identify CISTs other than those listed in Tables 13 and 14.

The expression of any number of Slcs, RTs, IRITs, NIRITs, GSTs, CISTs, or nucleic acids listed in Tables 1-14, 19, 20, 21, and 22 can be evaluated to determine whether or not transplanted tissue is injured. For example, the expression of one or more than one (e.g., two, three, four, five, six, seven, eight, nine, ten, 15, 20, 25, 30, 40, 50, 75, 100, or more than 100) of the nucleic acids listed in Tables 1-14, 19, 20, 21, and 22 can be used.

The term “elevated level” as used herein with respect to the level of a nucleic acid or polypeptide encoded by a nucleic acid listed in Tables 5-14 is any level that is greater than a reference level for that nucleic acid or polypeptide. For example, an elevated level of a nucleic acid or polypeptide encoded by a nucleic acid listed in Tables 5-14 can be about 0.3, 0.5, 0.7, 1.1, 1.2, 1.3, 1.4, 1.5, 1.6, 1.7, 1.8, 1.9, 2.0, 2.2, 2.4, 2.6, 2.8, 3, 3.3, 3.6, 4, 4.5, 5, 5.5, 6, 7, 8, 9, 10, 15, 20, or more times greater than the reference level for that nucleic acid or polypeptide, respectively.

The term “reduced level” as used herein with respect to the level of a nucleic acid or polypeptide encoded by a nucleic acid listed in Tables 1-4 is any level that is less than a reference level for that nucleic acid or polypeptide. For example, a reduced level of a nucleic acid or polypeptide encoded by a nucleic acid listed in Tables 1-4 can be about 0.3, 0.5, 0.7, 1.1, 1.2, 1.3, 1.4, 1.5, 1.6, 1.7, 1.8, 1.9, 2.0, 2.2, 2.4, 2.6, 2.8, 3, 3.3, 3.6, 4, 4.5, 5, 5.5, 6, 7, 8, 9, 10, 15, 20, or more times less than the reference level for that nucleic acid or polypeptide, respectively.

The term “reference level” as used herein with respect to a nucleic acid or polypeptide encoded by a nucleic acid listed in Tables 1-14 is the level of that nucleic acid or polypeptide typically expressed by cells in tissues that are free of injury. For example, a reference level of a nucleic acid or polypeptide can be the average expression level of that nucleic acid or polypeptide, respectively, in cells isolated from kidney tissue that has not been injured. In addition, a reference level can be any amount. For example, a reference level can be zero. In this case, any level greater than zero would be an elevated level.

Any number of samples can be used to determine a reference level. For example, cells obtained from one or more healthy mammals (e.g., at least 5, 10, 15, 25, 50, 75, 100, or more healthy mammals) can be used to determine a reference level. It will be appreciated that levels from comparable samples are used when determining whether or not a particular level is an elevated or reduced level. For example, levels from one type of cells are compared to reference levels from the same type of cells. In addition, levels measured by comparable techniques are used when determining whether or not a particular level is an elevated level or a reduced level.

Any suitable method can be used to determine whether or not a particular nucleic acid is expressed at a detectable level or at a level that is greater or less than the average level of expression observed in control cells. For example, expression of a particular nucleic acid can be measured by assessing mRNA expression. mRNA expression can be evaluated using, for example, northern blotting, slot blotting, quantitative reverse transcriptase polymerase chain reaction (RT-PCR), real-time RT-PCR, or chip hybridization techniques. Methods for chip hybridization assays include, without limitation, those described herein. Such methods can be used to determine simultaneously the relative expression levels of multiple mRNAs. Alternatively, expression of a particular nucleic acid can be measured by assessing polypeptide levels. For example, polypeptide levels can be measured using any method such as immuno-based assays (e.g., ELISA), western blotting, or silver staining.

The methods and materials provided herein can be used at any time prior to, during, or following tissue transplantation to determine whether or not the tissue is injured, rejected, or likely to be rejected. In some embodiments, a sample obtained from a donor at any time prior to transplantation can be assessed for the presence of cells expressing elevated levels of a nucleic acid listed in Tables 5-14, decreased levels of a nucleic acid listed in Tables 1-4, or significant alterations in gene profiles that correlate with genes listed Tables 1-14 (such as the gene profiles and pathways referred to in Tables 19, 20, 21, and 22). For example, a sample can be obtained from a donor 1, 2, 3, 4, 5, 6, 7, or more than 7 days prior to transplant, or can be obtained from a donor tissue within hours (e.g., 1, 2, 3, 4, 6, 8, or 12 hours) prior to transplantation. In some cases, a sample obtained from transplanted tissue at any time following the tissue transplantation can be assessed for the presence of cells expressing elevated levels of a nucleic acid listed in Tables 5-14, or decreased levels of a nucleic acid listed in Tables 1-4. For example, a sample can be obtained from transplanted tissue 1, 2, 3, 4, 5, 6, 7, 8, or more hours after the transplanted tissue was transplanted. In some cases, a sample can be obtained from transplanted tissue one or more days (e.g., 2, 3, 4, 5, 6, 7, 8, 9, 10, 15, 20, 25, 30, 40, 42, or more days) after the transplanted tissue was transplanted. Typically, a sample can be obtained from transplanted tissue 1 to 7 days (e.g., 1 to 3 days, or 5 to 7 days) after transplantation and assessed for the presence of cells expressing elevated levels of one or more IRITs, NIRITs, GSTs, or CISTs, expressing elevated levels of one or more nucleic acids listed in Tables 5-14, expressing decreased levels of one or more transcripts listed in Tables 1-4, or expressing significant alterations in gene profiles that correlate with genes listed Tables 1-14 (such as those gene profiles and/or pathways referred to in Tables 19, 20, 21, and 22).

In some cases, a mammal can be diagnosed as having transplanted tissue that is being rejected if it is determined that the mammal or tissue contains cells that express a reduced level of a cadherin polypeptide or a transporter polypeptide.

Any type of sample containing cells can be used to determine whether or not the mammal or transplanted tissue contains cells that express a reduced level of a cadherin polypeptide or a transporter polypeptide. For example, biopsy (e.g., punch biopsy, aspiration biopsy, excision biopsy, needle biopsy, or shave biopsy), tissue section, lymph fluid, blood, and synovial fluid samples can be used. In some embodiments, a tissue biopsy sample can be obtained directly from the transplanted tissue. In some embodiments, a lymph fluid sample can be obtained from one or more lymph vessels that drain from the transplanted tissue. A sample can contain any type of cell including, without limitation, cytotoxic T lymphocytes, CD4⁺ T cells, B cells, peripheral blood mononuclear cells, macrophages, kidney cells, lymph node cells, or endothelial cells.

Examples of cadherin polypeptides include, without limitation, E-cadherin polypeptides, Ksp-cadherin polypeptides, and any other cadherin polypeptide. Examples of transporter polypeptides include, without limitation, Slc2a2, Slc2a4, Slc2a5 Slc5a1, Slc5a2, Slc5a10, Slc7a7, Slc7a8, Slc7a9, Slc7a10, Slc7a12, Slc7a13, Slc1a4, Slc3a1, Slc1a1, aquaporins (e.g., aquaporin 1, aquaporin 2, aquaporin 3, and aquaporin 4), members of the family of ABC transporters, solute carriers, and ATPases.

The expression of any number of polypeptides disclosed herein or nucleic acids encoding such polypeptides can be evaluated to determine whether or not transplanted tissue will be rejected. For example, the expression of one or more than one (e.g., two, three, four, five, six, seven, eight, nine, ten, 15, 20, 25, 30, 40, 50, 75, 100, or more than 100) of the transporter polypeptides provided herein can be used. In some embodiments, determining that a polypeptide is expressed at a reduced level in a sample can indicate that transplanted tissue will be rejected. In some embodiments, transplanted tissue can be evaluated by determining whether or not the tissue contains cells that express one or more cadherin or transporter polypeptides at a level that is less than the average expression level observed in control cells obtained from tissue that has not been transplanted. Typically, a polypeptide can be classified as being expressed at a level that is less than the average level observed in control cells if the expression levels differ by at least 1-fold (e.g., 1.5-fold, 2-fold, 3-fold, or more than 3-fold). Control cells typically are the same type of cells as those being evaluated. In some cases, the control cells can be isolated from kidney tissue that has not been transplanted into a mammal. Any number of tissues can be used to obtain control cells. For example, control cells can be obtained from one or more tissue samples (e.g., at least 5, 6, 7, 8, 9, 10, or more tissue samples) obtained from one or more healthy mammals (e.g., at least 5, 6, 7, 8, 9, 10, or more healthy mammals).

Any appropriate method can be used to determine whether or not a particular polypeptide is expressed at a reduced level as compared to the average level of expression observed in control cells. For example, expression of a particular polypeptide can be measured by assessing mRNA expression. mRNA expression can be evaluated using, for example, northern blotting, slot blotting, quantitative reverse transcriptase polymerase chain reaction (RT-PCR), real-time RT-PCR, or microarray chip hybridization techniques. Methods for microarray chip hybridization assays include, without limitation, those described herein. Such methods can be used to determine simultaneously the relative expression levels of multiple mRNAs. Alternatively, expression of a particular polypeptide can be measured by assessing polypeptide levels. For example, polypeptide levels can be measured using any method such as immuno-based assays (e.g., ELISA and immunohistochemistry), western blotting, or silver staining.

The methods and materials provided herein can be used at any time following a tissue transplantation to determine whether or not the transplanted tissue will be rejected. For example, a sample obtained from transplanted tissue at any time following the tissue transplantation can be assessed for the presence of cells expressing a reduced level of a polypeptide provided herein. In some cases, a sample can be obtained from transplanted tissue 1, 2, 3, 4, 5, 6, 7, 8, or more hours after the transplanted tissue was transplanted. In some cases, a sample can be obtained from transplanted tissue one or more days (e.g., 2, 3, 4, 5, 6, 7, 8, 9, 10, 15, 20, 25, 30, 40, or more days) after the transplanted tissue was transplanted. For example, a sample can be obtained from transplanted tissue 2 to 7 days (e.g., 5 to 7 days) after transplantation and assessed for the presence of cells expressing a reduced level of a polypeptide provided herein. Typically, a biopsy can be obtained any time after transplantation if a patient experiences reduced graft function.

As described herein, a decreased expression of transcripts for many epithelium-specific transporters was found before the onset of tubulitis, indicating that the epithelium is an early target of the rejection process despite the fact that the lymphocytes have no apparent contact with the epithelium. In addition, the results provided herein demonstrate that the epithelium changes in response to rejection before tubulitis and independent of CD103, cytotoxic molecules, or antibody acting on the graft. Tubulitis and loss of cadherins in kidney allograft rejection can be associated with CD103 positive cells in the interstitium and epithelium, while not being dependent on CD103, and can be part of an ongoing tubulointerstitial process.

Ksp-cadherin mRNA and protein were decreased early, before the onset of tubulitis, coincident with interstitial infiltration. These results demonstrate that the decrease in Ksp-cadherin and E-cadherin can be attributed to the response of the epithelium to the inflammatory processes, responses that can permit the entry of inflammatory cells into the epithelium, and if unchecked can culminate in EMT.

While not being limited to any particular mode of action, one model of tubulitis can be as follows. T cell-mediated rejection in the interstitium can induce expression of effectors (e.g., TGF-β1, actins, vimentin, MMP2, collagens, hyaluronic acid, and many others) that can cause the tubule epithelium to change, permitting the interstitial inflammatory cells to enter the epithelium. The effector T cell/macrophage infiltrate can deliver this contact-independent signal to the epithelium via soluble factors or via matrix- or even microcirculation changes. The mechanism by which the interstitial CTL trigger epithelial changes can be that Tgfb1 plays a role. Tgfb1 is produced by CTL and is expressed in a CTL line and in recently generated allogeneic cultures, and potentially by macrophages and by many cells in the graft. The early increase in Tgfb1 in isografts can exaggerate in allografts, and some Tgfb1-inducible transcripts can be greatly increased in rejecting allografts. In addition, TGF-β1 can trigger a decrease in cadherin expression and alterations in epithelial function.

This description also provides nucleic acid arrays. The arrays provided herein can be two-dimensional arrays, and can contain at least 10 different nucleic acid molecules (e.g., at least 20, at least 30, at least 50, at least 100, or at least 200 different nucleic acid molecules). Each nucleic acid molecule can have any length. For example, each nucleic acid molecule can be between 10 and 250 nucleotides (e.g., between 12 and 200, 14 and 175, 15 and 150, 16 and 125, 18 and 100, 20 and 75, or 25 and 50 nucleotides) in length. In addition, each nucleic acid molecule can have any sequence. For example, the nucleic acid molecules of the arrays provided herein can contain sequences that are present within the nucleic acids listed in Tables 1-14, 19, and 20. For the purpose of this document, a sequence is considered present within a nucleic acid listed in, for example, Table 1 when the sequence is present within either the coding or non-coding strand. For example, both sense and anti-sense oligonucleotides designed to human Slc39a5 nucleic acid are considered present within Scl39a5 nucleic acid.

Typically, at least 25% (e.g., at least 30%, at least 40%, at least 50%, at least 60%, at least 75%, at least 80%, at least 90%, at least 95%, or 100%) of the nucleic acid molecules of an array provided herein contain a sequence that is (1) at least 10 nucleotides (e.g., at least 11, 12, 13, 14, 15, 16, 17, 18, 19, 20, 25, or more nucleotides) in length and (2) at least about 95 percent (e.g., at least about 96, 97, 98, 99, or 100) percent identical, over that length, to a sequence present within a nucleic acid listed in any of Tables 1-16. For example, an array can contain 100 nucleic acid molecules located in known positions, where each of the 100 nucleic acid molecules is 100 nucleotides in length while containing a sequence that is (1) 30 nucleotides in length, and (2) 100 percent identical, over that 30 nucleotide length, to a sequence of one of the nucleic acids listed in any of Tables 1-14, 19, and 20. A nucleic acid molecule of an array provided herein can contain a sequence present within a nucleic acid listed in any of Tables 1-14, 19, and 20, where that sequence contains one or more (e.g., one, two, three, four, or more) mismatches.

The nucleic acid arrays provided herein can contain nucleic acid molecules attached to any suitable surface (e.g., plastic or glass). In addition, any method can be use to make a nucleic acid array. For example, spotting techniques and in situ synthesis techniques can be used to make nucleic acid arrays. Further, the methods disclosed in U.S. Pat. Nos. 5,744,305 and 5,143,854 can be used to make nucleic acid arrays.

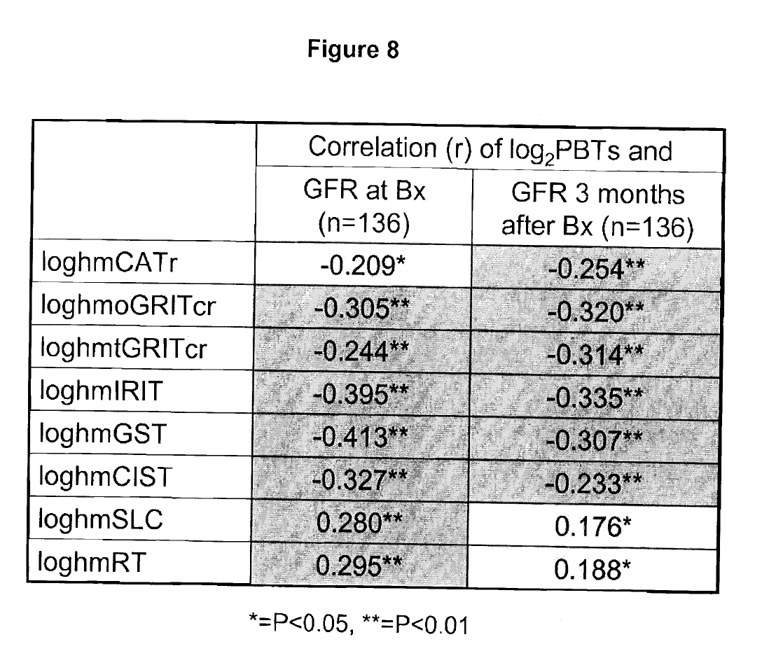

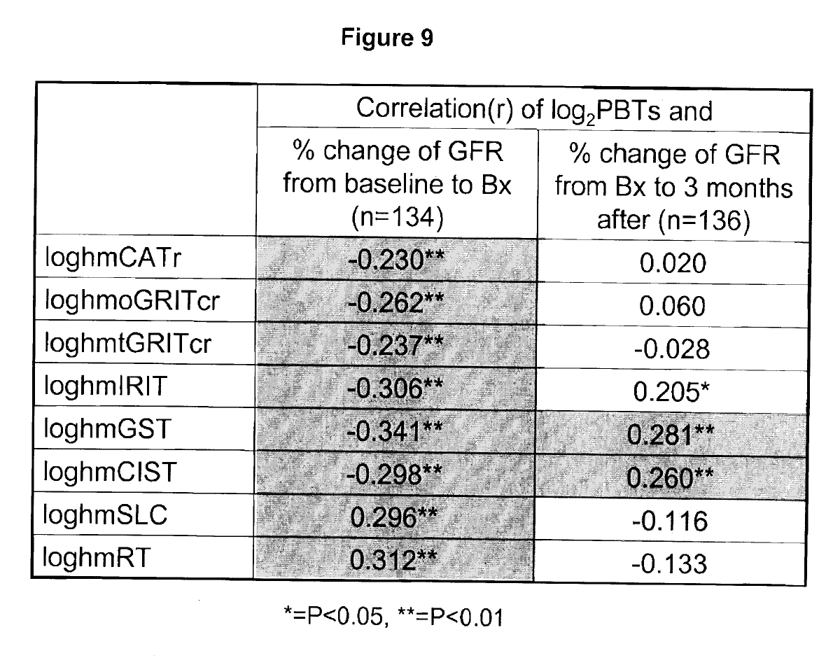

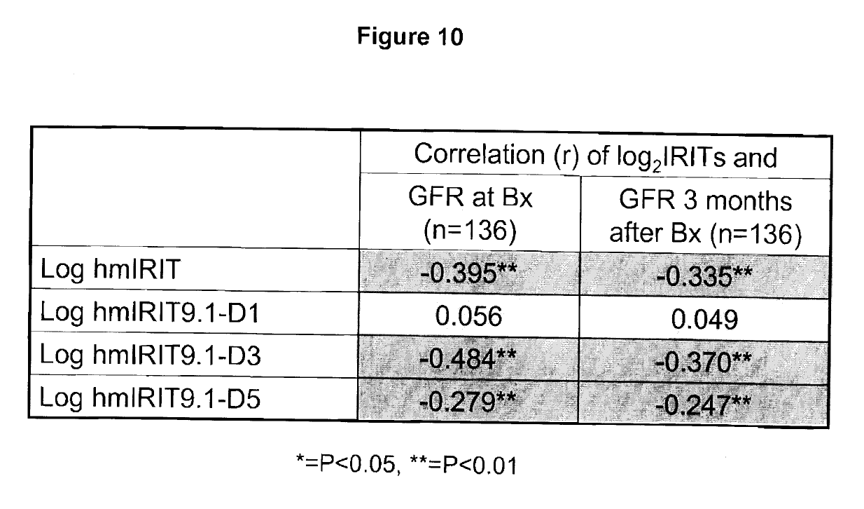

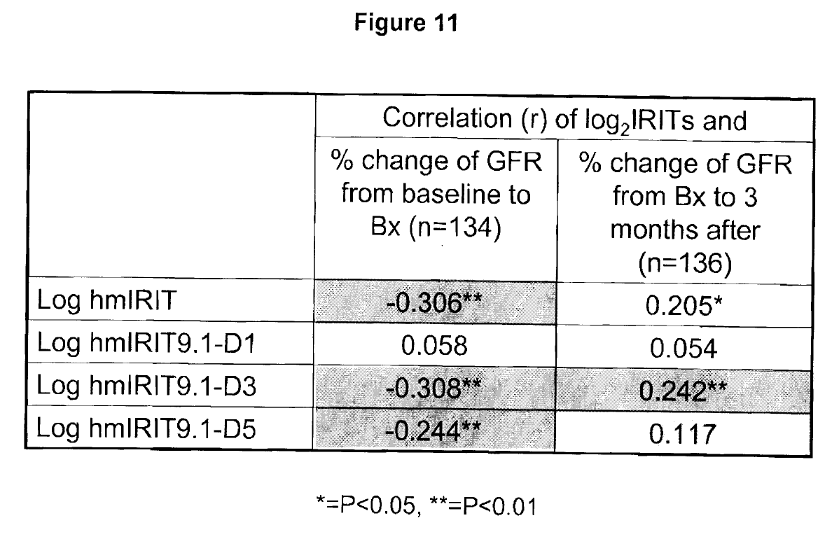

This description also provides methods and materials involved in determining the potential for recovery of organ function following injury. For example, FIG. 8 shows that the Slc, RT, IRIT, GST and CIST gene sets correlate with function (glomerular filtration rate; GFR) at the time of biopsy and at 3 months after the biopsy. FIG. 9 shows that gene sets correlate with the degree of loss of function/GFR before the biopsy (SLC's, RT's, IRITs, ST's, CISTs), as well as with recovery of function/GFR after the biopsy (IRITs, GSTs, CISTs). FIGS. 10 and 11 show that the best correlation between renal function and gene sets are with the IRITs, especially with IRITsD3 and IRITsD5 (refer to Table 7 (mouse) and Table 8 (human)).

This document also provides methods and materials to assist medical or research professionals in determining whether or not a tissue is injured, is at increased risk for developing DGF following transplantation, or is likely to recover from alloimmune or non-alloimmune injury. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of one or more nucleic acids or polypeptides encoded by nucleic acids listed in Tables 1-14, determining the level of a cadherin polypeptide, or determining the level of a transporter polypeptide in a sample, and (2) communicating information about that level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

### Computer-Readable Medium and an Apparatus for Predicting Rejection

This disclosure further provides a computer-readable storage medium configured with instructions for causing a programmable processor to determine whether a tissue that has been or is to be transplanted is injured, and/or to determine the potential for recovery of organ function. The determination of whether a tissue is injured can be carried out as described herein; that is, by determining whether one or more of the nucleic acids listed in Tables 5-14 and the third column of Table 20 is detected in a sample (e.g., a sample of the tissue), or expressed at a level that is greater than the level of expression in a corresponding control tissue, or by determining whether one or more of the nucleic acids listed in Tables 1-4 and the third column of Table 19 is expressed at a level that is less than the level of expression in a corresponding control tissue. In some cases, it can be determined whether a tissue is being rejected by determining whether or not the tissue contains cells that express a reduced level of a cadherin polypeptide or a transporter polypeptide. The processor also can be designed to perform functions such as removing baseline noise from detection signals.

Instructions carried on a computer-readable storage medium (e.g., for detecting signals) can be implemented in a high level procedural or object oriented programming language to communicate with a computer system. Alternatively, such instructions can be implemented in assembly or machine language. The language further can be compiled or interpreted language.

The nucleic acid detection signals can be obtained using an apparatus (e.g., a chip reader) and a determination of tissue injury can be generated using a separate processor (e.g., a computer). Alternatively, a single apparatus having a programmable processor can both obtain the detection signals and process the signals to generate a determination of whether injury is occurring or is likely to occur. In addition, the processing step can be performed simultaneously with the step of collecting the detection signals (e.g., “real-time”).

Any suitable process can be used to determine whether a tissue that has been or is to be transplanted is injured. In some embodiments, for example, a process can include determining whether a pre-determined number (e.g., one, two, three, four, five, six, seven, eight, nine, ten, 15, 20, 25, 30, 40, 50, 75, 100, or more than 100) of the nucleic acids listed in Tables 5-14 and the third column of Table 20 is expressed in a sample (e.g., a sample of transplanted tissue) at a level that is greater than the average level observed in control cells (e.g., cells obtained from tissue that has not been transplanted or is not to be transplanted, or in a control transplanted tissue). If the number of nucleic acids that are expressed in the sample is equal to or exceeds the pre-determined number, the tissue can be determined to be injured and the potential for recovery of organ/tissue function can be determined to be low, depending on the gene sets that are predominantly altered. If the number of nucleic acids that are expressed in the sample is less than the pre-determined number, the tissue can be determined not to be injured. The steps of this process (e.g., the detection, or non-detection, of each of the nucleic acids) can be carried out in any suitable order.

Also provided herein is an apparatus for determining whether a tissue that has been or is to be transplanted is injured. An apparatus for determining whether tissue injury has occurred can include, for example, one or more collectors for obtaining signals from a sample (e.g., a sample of nucleic acids hybridized to nucleic acid probes on a substrate such as a chip) and a processor for analyzing the signals and determining whether rejection will occur. By way of example, the collectors can include collection optics for collecting signals (e.g., fluorescence) emitted from the surface of the substrate, separation optics for separating the signal from background focusing the signal, and a recorder responsive to the signal, for recording the amount of signal. The collector can obtain signals representative of the presence of one or more nucleic acids listed in Tables 1-14, 19, and 20 (e.g., in samples from transplanted and/or non-transplanted tissue). The apparatus further can generate a visual or graphical display of the signals, such as a digitized representation. The apparatus further can include a display. In some embodiments, the apparatus can be portable.

The invention will be further described in the following examples, which do not limit the scope of the invention described in the claims.

### EXAMPLES

### Example 1

### Materials and Methods (Mouse Studies)

These studies utilized a mouse kidney allograft model that develops pathologic lesions that are diagnostic in human graft rejection. Basically, a comparison of mouse kidney pathology to the mouse transcriptome was used to guide understanding of the relationship of lesions to transcriptome changes in human rejection.

Mice: Male CBA/J (CBA) and C57B1/6 (B6) mice were obtained from the Jackson Laboratory (Bar Harbor, Me.). IFN-γ deficient mice (BALB/c.GKO) and (B6.129S7-IFNγ^{tmlTs}; B6.GKO) were bred in the Health Sciences Laboratory Animal Services at the University of Alberta. Mouse maintenance and experiments were in conformity with approved animal care protocols. CBA (H-2K, I-A^{k}) into C57B1/6 (B6; H-2 K^{b}D^{b}, I-A^{b}) mice strain combinations, BALB/c.GKO into B6.GKO were studied across full MHC and non-MHC disparities.

Renal transplantation: Renal transplantation was performed as a non life-supporting transplant model. Recovered mice were killed at day 1, 2, 3, 4, 5, 7, 14, 21 or 42 post-transplant. Kidneys were removed, snap frozen in liquid nitrogen and stored at −70° C. No mice received immunosuppressive therapy. Kidneys with technical complications or infection at the time of harvesting were removed from the study.

Acute Tubular Necrosis (ATN) model of ischemia reperfusion injury: The vascular pedicle of the left CBA kidney was clamped for 1 hour, kept moistened with PBS at 37° C. and then released. Animals were kept for 7 days and then sacrificed. The detailed procedure was previously published (Takeuchi et al. (2003) J. Am. Soc. Nephrol. 14:2823-2832). Kidneys representing the ATN model were denoted ATN D7. The histology of the ATN kidneys, in which ischemic injury was induced by cross-clamping 7 days earlier, was reported in detail elsewhere (Goes et al. (1995) Transplant 59:565-572). In brief, these kidneys showed severe acute tubular injury with flattening of tubular epithelium, variation in cell size and shape, cellular swelling, loss of PAS positive brush borders, and individual tubular epithelial cell necrosis with denudation of the epithelium from the basement membrane and shedding of granular cellular debris into the tubular lumen. In addition, tubular regenerative changes with nuclear enlargement, prominent nucleoli, and mitotic figures were observed. Kidneys with ATN also showed interstitial edema and a focal minimal interstitial mononuclear cell infiltrate.

Recombinant IFN-γ. rIFN-γ was a generous gift from Dr. T. Stewart at Genentech (South San Francisco, Calif.).

Microarrays: High-density oligonucleotide GeneChip 430A and 430 2.0 arrays, GeneChip T7-Oligo(dT) Promoter Primer Kit, Enzo BioArray HighYield RNA Transcript Labeling Kit, IVT Labeling KIT, GeneChip Sample Cleanup Module, IVT cRNA Cleanup Kit were purchased from Affymetrix (Santa Clara, Calif.). RNeasy Mini Kit was from Qiagen (Valencia, Calif.), Superscript II, E. coli DNA ligase, E. coli DNA polymerase I, E. coli RNase H, T4 DNA polymerase, 5× second strand buffer, and dNTPs were from Invitrogen Life Technologies.

RNA preparation and hybridization: Total RNA was extracted from individual kidneys using the guanidinium-cesium chloride method and purified RNA using the RNeasy Mini Kit (Qiagen). RNA yields were measured by UV absorbance. The quality was assessed by calculating the absorbance ratio at 260 nm and 280 nm, as well as by using an Agilent BioAnalyzer to evaluate 18S and 28S RNA integrity.

For each array, RNA from 3 mice was pooled. RNA processing, labeling and hybridization to MOE430 2.0 arrays was carried out according to the protocols included in the Affymetrix GeneChip Expression Analysis Technical Manual (available on the World Wide Web at affymetrix.com). cRNA used for Moe 430 2.0 arrays was labeled and fragmented using an IVT Labeling Kit and IVT cRNA Cleanup Kit.

Sample designation: Normal control kidneys were obtained from CBA mice and designated as NCBA. Allografts rejecting in wild type hosts (B6) at day 3 through day 42 post transplant were designated as WT D1, D2, D3, D4, D5, D7, D14, D21 and D42, respectively. Corresponding isografts were designated Iso D1, D2, D3, D4, D5, D7, D14, D21 and D42. Kidneys from mice treated with recombinant IFN-γ were designated rIFN-γ. BALB/c-GKO kidneys (deficient in IFN-γ) rejecting in IFN-γ-deficient B6 hosts at day 5 were designated as GKO D5, and corresponding isografts were designated ISO.GKO D5. All samples (each consisting of RNA pooled from 3 mice) were analyzed by the Moe 430 2.0 arrays in duplicates

Sample analysis: RMA-based method: raw microarray data was pre-processed using the RMA method (Bioconductor 1.7; R version 2.2). Microarrays (controls and treatments) were preprocessed separately for each mouse strain combination. After preprocessing, data sets were subjected to variance-based filtering i.e. all probe sets that had an inter-quartile range of less than 0.5 (log 2 units), across all chips, were removed. Filtered data was then used for transcript selection as follows: transcripts had to have a corrected p-value≧0.01, and had to be increased≧2-fold vs. appropriate controls. Corrected p-values were calculated using the “limma” package (fdr adjustment method), which uses an empirical Bayes method for assigning significance.

### Example 2

### Renal Transcripts (RTs) and Solute Carriers (Slcs)

The epithelium in mouse kidney allografts was examined for morphologic changes, and the relationship of such changes to immunologic effector mechanisms was defined. Rejecting allografts showed tubulitis, loss of epithelial mass, marked reduction of E-cadherin and Ksp-cadherin and redistribution to the apical membrane, indicating loss of polarity. Tubulitis and other morphologic changes in the epithelium were dependent on host T cells but independent of host perforin (Prfl), granzymes A and B (GzmA/B), CD103, and B cells. The changes in epithelial morphology likely reflect the effects of the T cell mediated interstitial inflammatory reaction, analogous to delayed type hypersensitivity (DTH).

Studies were conducted to explore the hypothesis that the T cell mediated inflammatory process in kidney allograft rejection induces major changes in renal parenchymal cells before histologic lesions such as tubulitis develop. Morphologic lesions (tubulitis, tubular shrinkage, loss of cadherins, and loss of polarity) may be a consequence and late manifestation of the epithelial response to the T cell mediated inflammatory process, which could be reflected in the transcriptome of renal parenchymal cells before histologic lesions develop. Microarrays were used to explore the early transcriptome changes of renal parenchymal cells in mouse allografts and isografts, their relationship to the evolution of histologic lesions such as tubulitis, and their relationship to immunologic effector mechanisms.

To analyze expression of transcripts that reflect changes in the epithelium, two sets of transcripts with high expression in normal kidney and low expression in inflammatory cells were selected. As a first set, epithelial transporters were selected because of their well documented importance for renal function. In particular, studies were focused specifically on the family of Slcs because of their extensive annotation. Members of the Slc family flagged “present” in normal kidney and “absent” (default conditions of GeneChip Operating Software 1.2, Affymetrix®) or with 5-fold lower expression in MLR, CTL, macrophages, fibroblasts, B cells, and CD8+ T cells compared to normal kidney were selected. If transcripts were represented by more than one probeset, the probeset with annotation “_at” and with the most robust signal in normal kidney was selected.

To extend the analysis to other RTs in an unbiased approach regardless of the gene family, all transcripts represented on the array were subjected to variance-based filtering (Bioconductor 1.7; R version 2.2); i.e., all probe sets with an inter-quartile range<0.5 (log 2 units) were removed (Bioinformatics and Computational Biology Solutions Using R and Bioconductor, 2005, Gentleman, Carey, Huber, Irizarry, and Dudoit, eds., Springer, New York). Of the remaining probesets, those with a signal>50 in all normal kidneys and 5× higher expression in normal kidney compared to MLR, CTL, CD8, B cells, primary macrophages, and fibroblasts (corrected p-value≦0.01) were selected. Corrected p-values were calculated using the “limma” package (FDR adjustment method; Smyth (2004) Stat. Appl. Genet. Mol. Biol. 3(1):Article 3).

The T cell infiltrate in allografts was detectable from day 1, and extended to the interstitium from days 5 to 7 post transplant, but morphologic epithelial changes did not develop until day 7. Transcripts for most Slcs were reduced in both allografts and isografts in response to transplant injury, but the loss was more severe and progressive in allografts and paralleled the development of tubulitis and other histologic lesions in the epithelium. Mouse Slcs are listed in Table 1; humanized versions of the mouse Slcs are listed in Table 2. Weighted sum decomposition of the Slc transcript set identified allospecific changes from day 1 and revealed multiple components of the allospecific epithelial response: sustained and progressive loss of transcripts, and lack of a positive response to injury. To assess whether specific functional subsets were affected by the loss of transcripts more than others, Slc subsets with specific biological functions (transporters of glucose, amino acids, organic ions, metal ions, Na, NaHCl, monocarboxyl acids, and mitochondrial transporters) were selectively analyzed. All subgroups showed a strikingly similar expression pattern in both isografts and allografts, respectively, resembling the pattern with loss of transcripts described earlier for the entire Slc set.

To derive a larger view of the effects of the alloimmune response on the kidney parenchymal cells, a more extensive set of renal transcripts (RTs) that was not restricted to specific gene families was defined (n=991; Tables 3 (mouse) and 4 (human)). Expression of RTs decreased post transplant, with more severe and progressive loss of transcripts in allografts compared to isografts, thus resembling the changes described for Slc transcripts.

Loss of transcripts was not attributable to simple dilution and affected the majority of renal transcripts, representing a selective structured program that leads to loss of at least some products and presumably function. The early changes in the transcriptome of renal parenchymal cells reflect the same mechanisms as the later development of histologic lesions such as tubulitis: loss of renal transcripts was dependent on the alloimmune response and T cells, but independent of IFN-K, Prfl, GzmA, GzmB, and alloantibody. The loss of epithelial transcripts should offer a system for objectively measuring the changes in renal allograft biopsies that can add to the current Banff system of grading morphologic lesions.

### Example 3

### (Not in Isografts) Injury and Repair Induced Transcripts (NIRIT)

The expression of genes during the alloresponse alone were investigated, excluding transcriptomes of infiltrating T cells, B cells and macrophages. Genes inducible by IFN-γ and genes activated in the isografts also were excluded.

First, all transcripts increased in at least one of the allograft conditions, i.e., day 1, 2, 3, 4, 5, 7, 14, 21, or 42 post transplant, were selected. This list then was corrected for IRIT (injury and repair induced transcripts—induced in the isografts), CAT (cytotoxic T cell associated transcripts), GRIT (gamma interferon dependent rejection induced transcripts), MAT (macrophage associated transcripts), BAT (B cell associated transcripts including immunoglobulin transcripts), and transcripts showing strain differences, using all probe sets corresponding to genes present in these lists. The final NIRIT list included 714 nonredundant genes (Table 5 lists the mouse genes; Table 6 lists the humanized versions of the mouse genes).

### Example 4

### Injury and Repair Induced Transcripts (IRIT)

Organs experience many stresses in the transplant procedure independent of the alloimmune response. To characterize the effects of these stresses on the organ, separately from allogeneic effects, global gene expression in mouse kidney isografts was studied. T cell-associated, macrophage associated, and IFN-γ inducible transcripts were excluded. Despite normal histology, expression of 970 “injury-and-repair inducible transcripts” (IRITs) was increased in isografts. Evaluation of host kidneys, acute tubular necrosis (ATN) model, and developing kidneys indicated that IRITs represent footprints of systemic stress, acute tubular injury and dedifferentiation. IRITs showed enrichment in GeneSpring Gene Ontology (GO) categories related to morphogenesis, extracellular matrix, response to stress and cell cycle. The expression pattern of IRITs showed significant correlations with the KEGG pathways, including TGFβ signaling, apoptosis, and cell cycle.

Using K-means clustering, the time course of IRIT expression was de-convoluted into three profiles, designated IRIT-D1, IRIT-D3 and IRIT-D5, which were characterized by peak expression in particular days post-transplant (refer to Table 7). The IRIT-D1 profile showed enrichment in systemic response and epithelium development, and IRIT-D3 showed enrichment in stress response, epithelium development, and mesenchyme differentiation, while IRIT-D5 represented stress response, extracellular matrix, cell cycle, TGFβ signaling, epithelial development, and mesenchyme differentiation. Thus, injury from transplant procedures can induce multiple transcriptional programs that reflect healing and repair, which eventually resolve. It is striking that genes representing different pathways share similar expression profiles, implying an orchestrated response to stress.

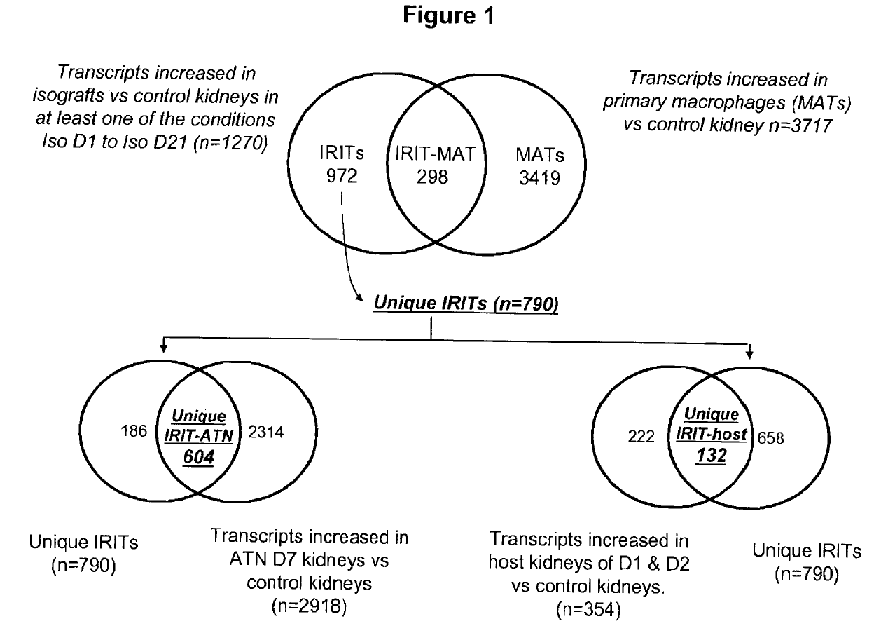

The algorithm for identifying IRITs is shown in FIG. 1. Transcripts that were over-expressed in the isografts at days 1-21 post-transplant were selected. This list was corrected for CAT, GRIT, MAT (670, 567 and 3717, respectively), and transcripts showing strain differences (Famulski et al. (2006) Am. J. Transplant. 6:1342-1354), using all probe sets corresponding to genes present in these lists. This selection yielded 790 unique IRIT (Table 7) that were elevated in the isografts and, most probably, represent kidney cell expression. Humanized versions of the mouse IRITs are listed in Table 8.

For identification of primary macrophages associated transcripts (MATs), the microarray data was analyzed by the GCOS method (Famulski et al., supra). Transcripts were required to be flagged as present, increased≧5-fold over the NB6 kidneys in at least one of the culture conditions, and have ae raw signal in NB6 and NCBA kidney below 200. The resulting list contained 2140 redundant transcripts. The total number of probe sets corresponding to genes present in this list was 3717.

Through this analysis, elevated expression of an additional 243 IRIT transcripts was attributed to macrophages present in the grafts (Table 9). Genes induced in the ATN model, which is a mouse model for ischemia reperfusion injury also were studied, and those that overlapped with IRITs were selected. As many as 604 transcripts were found in the IRITs list, and were defined as IRIT-ATN (Table 7). Thus, isografts demonstrated gene expression that is highly comparable to that in the ATN model, despite their normal histology. The top 25 IRITs differentiating allografts from isografts at day 1, day 2, day 3, day 4, day 5, day 7, and day 21 are listed in Table 10.

The systemic effect of graft transplantation on IRITs expression also was studied by analyzing IRIT expression in iso-host D1 and D2 kidneys. One hundred and twenty-nine IRIT-host transcripts were identified that were expressed both in the isografts and in the host kidneys. Expression of these genes probably reflects the systemic effects of surgical procedure. Expression of an additional 17 transcripts was attributed to macrophages. IRITs were annotated using the GO terms. Excluding the parent terms, IRITs were significantly overrepresented in biological processes such as response to stress (including response to wounding and wound healing), cell cycle and cell proliferation, cell communication including cell adhesion, organ development, and morphogenesis. IRITs also were highly represented in extracellular matrix components (including collagens), cytoskeleton and cell junctions.

Studies then were conducted to investigate which pathways correlate with the IRITs expression profile in isografts at days 1-21. Spearman correlation of IRIT expression profile with the MAPP and KEGG pathways demonstrated high similarity (≧0.75) of 27 pathways, including apoptosis, cell cycle regulation, and TGFβ signaling. Interestingly, the IRIT expression profile showed a high negative correlation (−0.75) with epithelial transporters. Prompted by enrichment of the GO categories related to morphogenesis and organ development, published expression data sets derived from developing kidneys were reanalyzed and compared with the IRITs (Schmidt-Ott et al. (2005) J. Am. Soc. Nephrol. 16:1993-2002; Schwab et al. (2003) Kidney Int. 64:1588-1604.) Genes involved in kidney development were derived using three comparisons: E12.5 metanephron mesenchyme vs E12.5 uteretic bud (combined stalk and tip), E11.5 metanephron mesenchyme vs adult kidney, and combined embryonic kidney tissues stages E11.5, E12.5, E13.5 and E16.5 vs. adult kidney, excluding metanephron mesenchyme. The IRITs expressed during development were identified using the nonredundant IRITs list and all probe sets corresponding to genes identified in developing kidneys. Eighty four IRITs were identified in E12.5 metanephron mesenchyme vs. E12.5 uteretic bud, 88 IRITs were identified in E12.5 uteretic bud vs E12.5 metanephron mesenchyme, 65 in combined embryonic kidney tissues stages vs. adult kidney (excluding mesenchyme), and 67 in E11.5 metanephron mesenchyme vs. adult kidney.

### Example 5

### Gamma Interferon Suppressed Transcripts (GST)

Interferon-gamma (IFN-γ) has a surprising protective effect in organ allografts, in that mouse kidney allografts lacking IFN-γ effects manifest accelerated congestion and necrosis. To understand this protection, histology, inflammatory infiltrate, and gene expression were assessed in IFN-γ receptor-deficient kidney allografts transplanted into wild-type and various knockout hosts. Early congestion and necrosis in the IFN-γ receptor-deficient allografts was unchanged in B cell deficient hosts, but was completely abrogated in hosts deficient either in perforin or in granzymes A and B. Thus, congestion and necrosis was independent of antibody but was completely dependent on host perforin and granzymes A and B. Many features of inflammation were altered, with increased neutrophils and increased transcripts for interleukin-4 (IL-4) and interleukin-13 (IL-13). Microarray analysis revealed increased expression of many IFN-γ-suppressed transcripts associated with alternative macrophage activation, including arginase 1, matrix metalloproteinase 9, and mannose receptor. The altered inflammation was independent of antibody and largely independent of host perforin or granzymes A and B. Thus, in kidney allografts, IFN-γ acts through the donor IFN-γ receptors to induce signal that determines which effector mechanisms act in the allograft, inhibiting perforin-granzyme-mediated congestion and necrosis and suppressing alternative inflammation.

The transcriptomes of allografts deficient in IFN-γ signaling were compared to WT allografts and normal kidneys. The resulting transcript lists then were corrected for CAT, GRIT, and transcripts showing strain differences, using all probe sets corresponding to genes present in these lists. Two hundred and seventeen non-redundant genes were identified that were over-expressed in IFN-γ-deficient allografts (Table 11; humanized versions of the mouse genes are listed in Table 12). The GST list was inspected for the most overrepresented categories. After excluding parent categories, GO subcategories containing at least five GSTs included: response to stress (including response to wounding), cell adhesion, peptidase activity (including metalloendopeptidase activity), and extracellular matrix components. Genes associated with the response to stress/wounding included highly expressed Chi313, F13a1 and Fgg. Genes related to peptidase activity included members of the Mmp (e.g., Mmp9, Mmp12), Adam, and Serpin families. Cell adhesion process genes included genes associated with pattern recognition, e.g., Mgl1 and C type lectins (Clec family members 1, 4, 7), and Thbs1. Extracellular matrix components included collagens Col3a1 and Col5a2, and Timp1. GO annotations of GSTs are shown in Table 11. The most highly expressed GSTs in terms of fold increase were those associated with alternative macrophage activation (AMA), i.e., Arg1, Chi313, Mmp12, and other macrophage and/or neutrophil activities (S100a8, S100a9 and Ear11). Additional AMA markers among the GSTs were Ear2, Mgl1, Mmp9, Mrc1, and Thbs1. The top 30 GSTs included IL-6 and chemokines Cxc12, Cxc14, Cxc17, Ccl6, Ccl24. Expression of plasminogen activator inhibitors Serpinb2 and Serpine1 also was very high. Thus, the GSTs include genes involved in the macrophage response to activation, proteolysis, response to wounding, and cell adhesion. At least 64 GSTs were associated with kidney necrosis (i.e., their expression was significantly decreased when the necrosis of IFN-K receptor-deficient allografts was averted). The most decreased GSTs were Serpinb2, Cxc17 and Clec1b. Many of the decreased GSTs are known to be involved in response to stress, injury, and tissue repair (e.g., adrenomedullin/Adm, heme oxygenase/Hmox1, I16, fibulin/Fbln2, tenascin/Tnc and thrombospondin1/Thbs1, Serpinb2, and Serpine1).

### Example 6

### Class I Suppressed Transcripts (CIST)

In mouse kidney allografts, IFN-γ acting on allograft IFN-γ receptors induces a signal that prevents early congestion and necrosis and determines inflammatory phenotype as the alloimmune response develops. It was hypothesized that this signal may be high expression of donor MHC class Ia and Ib proteins, which have the potential to control host infiltrating cells via inhibitory receptors. Thus, it was postulated that class I-deficient allografts should resemble IFN-γ receptor deficient allografts. Two types of class I deficient allografts were studied: Tap1 transporter-deficient or beta 2 microglobulin-deficient, transplanted into wild-type hosts. Although many IFN-γ-induced transcripts were increased, class I-deficient allografts developed congestion and necrosis between days 5 and 7, similar to IFN-γ receptor-deficient allografts. Expression of TH2 cytokines IL-4 and IL-13 also was increased, despite abundant IFN-γ expression. Microarray analysis of gene expression identified 78 transcripts elevated in class I-deficient allografts that were previously identified as elevated in IFN-γ-deficient allografts, including many markers of alternative macrophage activation (e.g., arginase 1). Thus, it was proposed that in organ allografts, elevated expression of donor class I induced by IFN-γ delivers an inhibitory signal to host inflammatory cells that prevents early graft necrosis, and also prevents some TH2 type inflammatory features.

The transcriptomes of Tap1KO and B2mKO allografts at day 7 were compared to WT (B6) allografts at day 7 and normal B6 control kidneys. These lists were then corrected for CAT, GRIT, and transcripts showing strain differences, using all probe sets corresponding to genes present in these lists. Seventy-eight unique genes were significantly over-expressed in both types of class I-deficient allografts. These were designated as the “class I suppressed transcripts” (CISTs; Table 13, with humanized versions of the mouse genes listed in Table 14).

The CIST list was analyzed using the GO browser. After excluding parent categories, GO subcategories containing at least 3 CISTs included: response to external stimulus (including Cxc14, Cxc17, I16, Hmox1, F7 and F13a1), angiogenesis (e.g., Thbs1), cellular catabolism (e.g., Arg1), endopeptidase activity (including Mmp12, Serpine1 and Serpinb2), and carbohydrate binding (e.g., Mrc1). Many CISTs were associated with the extracellular space, including members of the Mmp and Adam families. The 30 most increased CISTs included Serpinb2, Mmp12, Arg1, interleukins (IL-6, IL-11), and chemokines (Cxc14, Cxc17). Some CISTs had been described as macrophage associated. Indeed, it was found that 32 CISTs were highly expressed in primary macrophages, including alternative macrophage activation (AMA) markers, e.g., arginase1 (Arg1), mannose receptor1 (Mrc1), and Mmp12. Others were linked to both neutrophils and macrophages (e.g., S100a8 and Ear11). Thus, CISTs represent genes involved in macrophage activation, with activities including proteolysis, angiogenesis, and extracellular matrix remodeling.

Overlap between the CISTs and the GSTs (transcripts over-expressed in GRKO allografts) was observed. Of 78 unique CISTs, 56 were increased in GRKO allografts day 7. Thus, expression of many transcripts, including Arg1, Mmp12, Mrc1, and Thsb1 can be elevated either when the graft lacks IFN-γ signaling, or has decreased expression of class I in the presence of IFN-γ.

### Example 7

### Other Gene Sets and Pathways Significantly Correlate with the Orchestrated Response Depicted by the Gene Profiles Listed in Tables 1-14

Gene profiles and pathways that significantly positively or negatively correlate with the gene sets listed in Tables 1-14 were identified as follows.

Table 19: the Slc score (the geometric mean of the ratios of each Slc probeset to that probeset's average value in the 8 controls) for each of the 143 biopsy for cause samples was calculated. The correlation between these 143 values and the 143 scores (again, sample expression to control average expression ratio) for each probeset on the array was calculated. This set of 54,675 correlations was then ordered. Genes with more than one probeset were reduced to a single probeset—that with the highest absolute value for a correlation. All probesets for genes included in the Slc set, as well as unannotated probesets, were removed. Of the remaining probesets, those with the 25 most positive and 25 most negative correlations were selected.

Table 20: The IRIT score (the geometric mean of the ratios of each IRIT probeset to that probeset's average value in the 8 controls) for each of the 143 biopsy for cause samples was calculated. The correlation between these 143 values and the 143 scores (again, sample expression to control average expression ratio) for each probeset on the array was calculated. This set of 54,675 correlations was then ordered. Genes with more than one probeset were reduced to a single probeset—that with the highest absolute value for a correlation. All probesets for genes included in the IRIT set, as well as unannotated probesets, were removed. Of the remaining probesets, those with the 25 most positive and 25 most negative correlations were selected

Table 21: All KEGG pathways represented by more than 5 probesets on the chips were selected. Scores for each KEGG pathway were calculated in the same way as were the Slc scores. The correlation between the Slc scores and each of the 177 KEGG scores (across all 143 biopsies for cause) was calculated. This set of 177 correlations was then ordered. The KEGG pathways with the 25 most positive and 25 most negative correlations were selected.

Table 22: All KEGG pathways represented by more than 5 probesets on the chips were selected. Scores for each KEGG pathway were calculated in the same way as were the IRIT scores. The correlation between the IRIT scores and each of the 177 KEGG scores (across all 143 biopsies for cause) was calculated. This set of 177 correlations was then ordered. The KEGG pathways with the 25 most positive and 25 most negative correlations were selected

The gene set in Table 19 and the gene pathways in Table 21 correlate with the gene profile shown in Tables 1 and 2 (mouse and human Slcs), while the gene set in Table 20 and the gene pathways in Table 22 correlate with the gene profile in Tables 7 and 8 (mouse and human IRITs).

### Example 8

### Materials and Methods (Human Studies)

Patients and clinical data: Implant biopsies for transcriptome analysis were obtained by taking 18 gauge core samples from donor kidneys. Donor data were collected retrospectively and recipient data prospectively. Renal allografts were biopsied intra-operatively within one hour of revascularization. One core was sent for routine histology. An additional core sample was immediately placed into RNAlater® (Qiagen) for subsequent RNA extraction. All biopsies were read using conventional renal histopathologic techniques and scored according to the Banff classification (Racusen et al., supra) by two independent renal histopathologists.

Delayed graft function (DGF) was defined as the need for dialysis (RRT) within the first week after transplantation. The decision to initiate dialysis was at the discretion of the primary transplant nephrologists and transplant surgeons, with no involvement of study investigators. Known risk factors for poor post-transplant function were defined based on extended donor criteria, and other factors predisposing to acute kidney injury (Schold et al. (2005) Am. J. Transplant. 5(4 Pt 1):757-765; Swanson et al. (2002) Am. J. Transplant. 2:68-75; Port et al. (2002) Transplant. 74:1281-1286; Nyberg et al. (2003) Am. J. Transplant. 3:715-721; Ojo et al. (1997) Transplant. 63:968-974; Grossberg et al. (2006) Transplant. 81:155-159; Randhawa (2001) Transplant. 71:1361-1365; and Remuzzi et al. (1999) J. Am. Soc. Nephrol. 10:2591-2598). These risk factors included: donor age≧60 years; percent sclerosed glomeruli (% SG)≧20%; cold ischemia time (CIT)≧24 hours; revascularization time (RVT)≧45 minutes; intra-operative mean arterial pressure (MAP)≦70 mmHg; surgical complications (vasospasm, mottled kidney, and delayed pinking/turgidity); cerebrovascular accident (CVA) as cause of death; donor creatinine≧130 μmol/L; donor large vessel atherosclerosis; renal histopathologic features of fibrointimal thickening and/or vascular disease (as a surrogate marker of donor hypertension); and other renal pathology present on biopsy. Individual donor kidney histologic scores were calculated based on the global kidney score (GKS) system (Remuzzi et al, supra).

RNA preparation and amplification: Total RNA was isolated using the RNeasy® Mini Kit (QIAGEN, Valencia, Calif.), and amplified according to Affymetrix® protocol (Santa Clara, Calif.) protocol. If the starting input of cRNA was below 2.5 μg, an additional round of linear amplification was conducted. RNA yields were measured by UV absorbance and RNA quality assessed by Agilent Bioanalyzer.

Microarray processing: RNA labeling and hybridization to the Affymetrix® GeneChip microarrays (human Hu133 Plus 2.0) was carried out according to the protocols included in the Affymetrix® GeneChip Expression Analysis Technical Manual.

Analysis of the transcriptome and clinical data: All sample chips, as well as eight nephrectomy controls (for calculating PBT scores) were pooled into one normalization batch and preprocessed using robust multi-chip averaging (RMA), implemented in Bioconductor version 1.7, R version 2.2. An inter-quartile range (IQR) cutoff of 0.5 log 2 units was then used to filter out probe sets with low variability across the entire dataset. Hierarchical clustering and principal components analysis (PCA) were then used to discover clusters within the dataset without any a priori sample classification. Biological pathways were identified using the KEGG-library (Kanehisa et al. (2006) Nucl. Acids Res. 34: 354-357; or World Wide Web at genome.adjp/kegg/).

Pathogenesis based transcript sets (PBTs) were tested in relation to differentiation of the various groups of implant samples derived from the unsupervised clustering methods. The selected PBTs included CATs (reflecting T cell burden), GRITs (reflecting IFN-K effects, IRITS and NIRITs (reflecting injury and repair in isografts and allografts, and RTs as well as Slcs (reflecting epithelial integrity of the kidney organ).

Standard class comparison methods were used to compare known classes in search of differentially expressed genes. All “adjusted p-values” reported refer to false discovery rates (fdr), e.g., an adjusted p-value of 0.01 signifies that 1% of the probe sets identified as significant at the 0.01 level will, on average, be false positives.

Among the different patient groups, dichotomous variables were compared using the Chi-square test. Continuous variables were compared using the t-test for those variables which were approximately normally distributed, and the nonparametric Mann-Whitney U test for those that were not normally distributed. Glomerular filtration rate (GFR) was estimated using Cockroft Gault equation: (140−R age)*R lean body weight*R gender)/(72*R crea*0.0113).

### Example 9

### Unsupervised Transcriptome Analysis

Eighty-seven consecutive implant biopsies were included in these studies: 42 from 31 deceased donors (DD), including 11 pairs, and 45 from living donors (LD). Of the 42 DD transplant recipients, 10 had DGF, whereas 1 of 45 LD recipients experienced DGF (p=0.003). The mean duration of follow up was 411±188 days. During follow up, two patients with functioning grafts died, and no further grafts were lost, giving a graft survival rate of 97.7%.

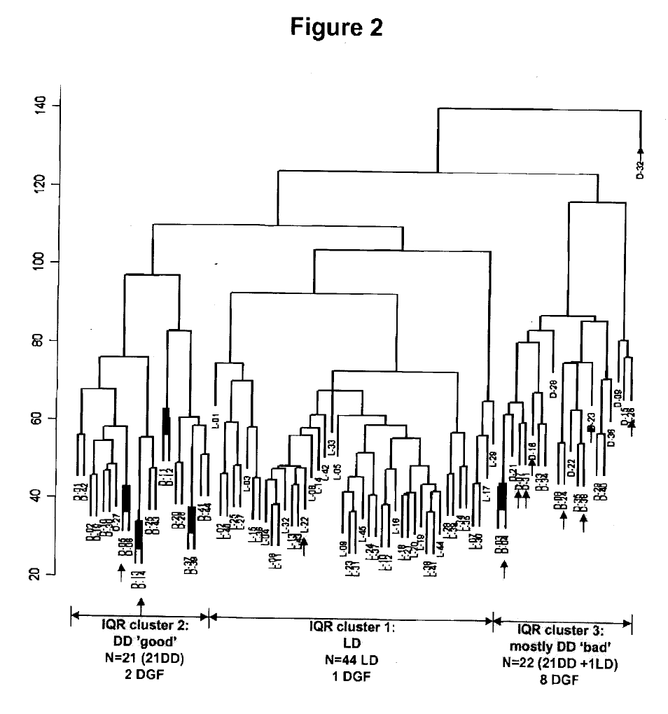

From the 54675 probesets represented on the microarray, 7376 probe sets passed the IQR filter. Unsupervised hierarchical cluster analysis of these 7376 probe sets, using DIANA, revealed two major clusters and one solitary outlier (FIG. 2). Interestingly, despite the unsupervised nature of the analysis, kidneys were clustered depending on donor origin: the larger cluster on the left was comprised of two subclusters, Cluster 1 (44 LD kidneys) and Cluster 2 (21 DD kidneys); the cluster on the right, Cluster 3, included 21 DD and 1 LD kidney. One patient in Cluster 1 experienced DGF (2.3%), compared to 2 in Cluster 2 (9.5%) and 8 in Cluster 3 (36.4%). The incidence of DGF was significantly different between Clusters 1 and 3 (p≦0.001) and between Clusters 2 and 3 (p≦0.05). The incidence of DGF was not significantly different between Clusters 1 and 2.

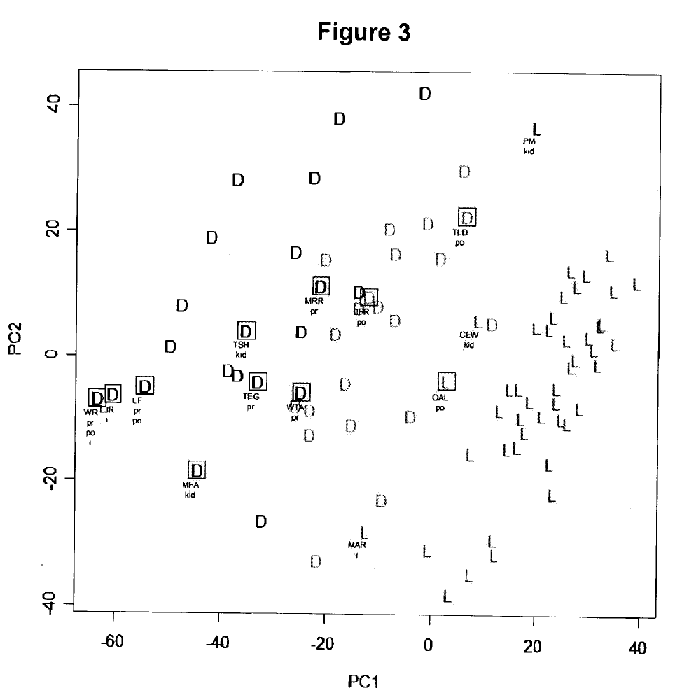

The same set of 7376 IQR filtered probesets was subjected to a further unsupervised principal component analysis (PCA). PCA showed strong grouping of LD versus DD kidneys (FIG. 3). There was wider scatter within the DD group, indicating greater heterogeneity among the samples. Clusters 2 and 3 were observed to form a continuum across the space of the first two principal components. The single outlier identified in FIG. 2 lies to the most extreme left in FIG. 3. This patient had the worst outcome of all 87 patients, requiring RRT for 2 months post-transplantation. Thus, both independent methods of unsupervised analysis revealed a good separation of LD from DD samples, indicating that the gene expression pattern seen in the DD samples is associated with function. Thus, the transcripts detect the difference between LD and DD, and detect significant heterogeneity among DD.

### Example 10

### Clinical Characteristics and Functional Outcomes

The demographics and clinical characteristics of all LD and DD implants are outlined in Table 17. Major differences between LD and DD groups included: more female donors in LD (p=0.004); greater HLA mismatches in DD (p<0.001); and longer cold ischemia time in DD (p<0.001). DD kidneys had a greater percent sclerosed glomeruli compared to LD (p=0.037). The global kidney score was higher in DD versus LD kidneys (p=0.036). Overall, 26 kidneys had a global kidney score≧4 (18 DD, 8 LD). As expected, the incidence of DGF was significantly greater in DD kidneys (p=0.003). Among all DD, the significant differences between patients with DGF versus those with IGF included higher recipient age in DGF (p=0.002), fewer HLA mismatches in DGF (p=0.009), and longer revascularization time in DGF (p=0.039). There were no significant differences in other clinical variables, including donor age and gender. Subsequent acute rejection rates and CMV episodes were not different between DGF and IGF groups.

Between the two clusters of DD kidneys, DGF was significantly greater in Cluster 3 (p=0.03). Serum creatinine was significantly higher in Cluster 3 versus Cluster 2 at day 7 (p=0.008). When patients requiring RRT were excluded, however, day 7 creatinine remained higher in Cluster 3, but was not statistically significant (p=0.103). Thus, the heterogeneity detected by the transcripts corresponds with differences in early function.

The differences between these two clusters of DD kidneys were examined to understand the significance of the heterogeneity in the DD. The single LD kidney in Cluster 3 was omitted, to focus exclusively on DD samples. There were no major differences in donor and recipient characteristics between Clusters 2 versus 3, with the exception of more female donors in Cluster 3 (p=0.011). Clinical factors including cold ischemia time, revascularization time, and intra-operative mean arterial pressure were not different. Furthermore, the percent sclerosed glomeruli and the global kidney score were not different. This confirms that the transcript differences were above and beyond any known clinical differences in these kidneys.

The number of renal risk factors experienced by patients in Clusters 2 and 3 were analyzed to assess whether these may explain the differences in clinical outcome (Table 18). The number of patients experiencing renal risk factors in Clusters 2 and 3 was not different (n=17 Cluster 2, n=19 Cluster 3). Among all patients with risk factors, the incidence of DGF was significantly greater in Cluster 3 (p≦0.05). This observation suggests enhanced susceptibility to DGF in Cluster 3, despite remarkable similarity of multiple clinical and histological variables with Cluster 2. Cluster 3 therefore constitutes a ‘high risk’ and Cluster 2 a ‘low risk’ group for DGF. By 12 months of follow-up, there were no observable differences in renal function between LD versus DD kidneys or between Clusters 2 and 3. Thus, certain transcripts permit an assessment of probability of good early function versus impaired early function.

### Example 11

### Transcripts Differentially Expressed Between DD and LD

In a comparison between DD and LD samples, 3718 probe sets were found to be differentially expressed at an fdr of 0.01. Altogether, 1929 probesets showed a significantly higher expression in DD vs LD samples, and 1789 probesets a significantly lower expression in DD vs LD samples. Transcripts most significantly increased in DD versus LD included fibrinogens FGG, FGB, and FGA; serine proteinase inhibitors SERPINA3 and SERPINA1; lactotransferrin, LTF; superoxide dismutase, SOD2; and lipopolysaccharide binding protein, LBP. These transcripts were more than 5-fold higher in DD samples. Others included complement components C6, C3, C1R, C1RL; chemokines CXCL2, CXCL1, CXCL3, CCL3, and IL8. Transcripts reduced in DD versus LD kidneys included many related to metabolism of fatty acids and amino acids (lysine, serine, threonine, tryptophane, arginine, proline and alanine); members of the albumin gene family (albumin, ALB; afamin, AFM; group-specific component, GC); and transporters (e.g. amino-acid transporter SLC7A13, the probe set with the lowest transcript level in DD versus LD).

### Example 12

### Transcripts Differentially Expressed Between ‘High Risk’ and ‘Low Risk’ DD Kidneys

Between Clusters 3 and 2, 1051 probe sets (‘High Risk-Low Risk’ set), were differentially expressed at an fdr of 0.01:404 probesets were increased and 647 decreased in Cluster 3 vs. Cluster 2. Transcripts demonstrating higher expression in the ‘High Risk’ versus ‘Low Risk’ groups included genes associated with the immunoglobulin family, e.g., IGKC, IGKV1-5, IGLJ3, IGHG3, IGHG1; collagens and integrins; chemokines including CCL2, 3, 4, 19, and 20; Toll-like receptor signaling, including CCL3, 4, STAT1, Ly96, and CD14; antigen processing and presentation, including HLA-DQA1, HLA-DQB1, HLA-DPA1; and renal injury markers such as HAVCR1 (KIM-1). Transcripts demonstrating lower expression in the ‘High Risk’ versus ‘Low Risk’ groups predominantly included genes related to glucose, fatty acid, and amino acid metabolism.

### Example 13

### Genes Associated with Outcomes

Studies were conducted to determine how many genes were significantly associated with the differences between LD and DD, between DD cases in cluster 2 and cluster 3, and between DD cases with DGF and IGF. Surprisingly, it was found that many (3718) probesets differed between DD and LD, and 1051 between DD in cluster 2 (low risk) versus cluster 3 (high risk) (fdr of 0.01). Many of the genes separating these kidneys had previously been identified in the PBT gene sets described herein and in the other patent applications referred to in this document. Thus, the genes separating DD from LD and high risk DD from low risk DD were the genes previously identified as IRITs, NIRITS, mCATs, GRITs, GSTs, CISTs, RTs, and Slcs.

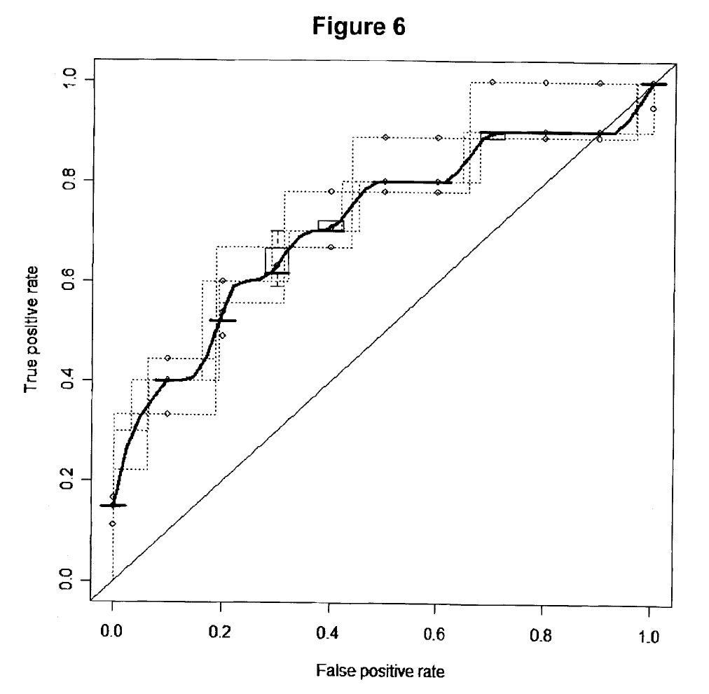

To determine whether such genes could predict the risk of DGF in a particular kidney, Receiver Operating Characteristic (ROC) analysis performed for Principal Component 1 (PC1) was compared to ROC performed for LD-DD and for cluster 2 vs. cluster 3 genes. PC1 was based on all probesets that passed the IQR-filter, and on all 87 (LD+DD) samples. The ROC curve shown in FIG. 6 indicates the value of PC1 in predicting DGF status in the 42 DD kidneys.

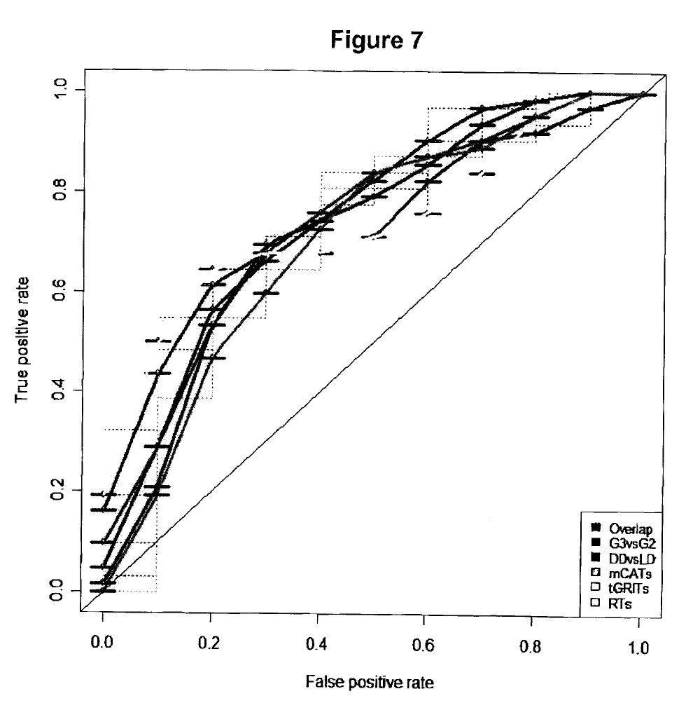

FIG. 7 shows ROC curves for individual PBT scores (RTs, tGRITs, mCATs) or PC1 scores in predicting DGF status in the 42 DD kidneys. The PC1 scores were based on PBTs and on genes that were IQR filtered.

### Example 14

### Many Genes in the LD vs. DD and Cluster 2 vs. Cluster 3 Genes Sets are Members of Previously Identified Pathogenesis Based Transcript Sets (PBTs)

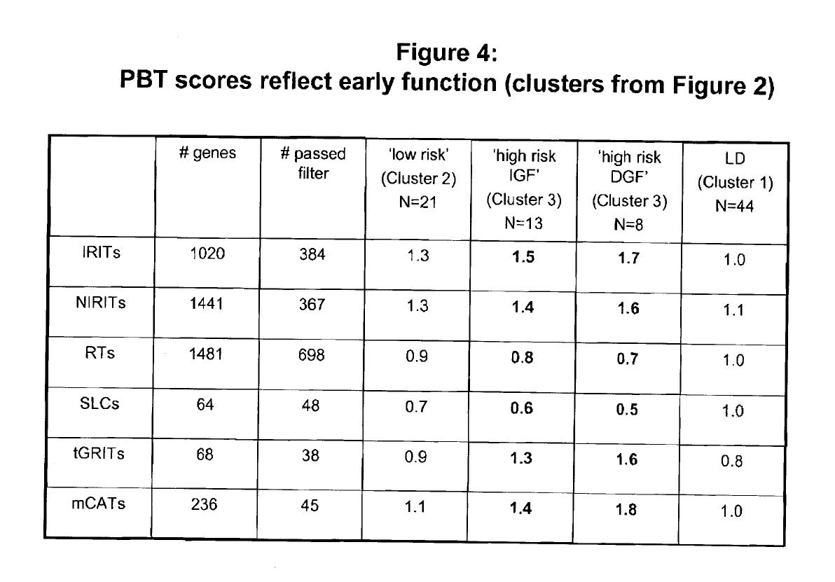

A large proportion of the transcripts in both the DD vs LD and High Risk vs Low Risk sets (clusters 3 vs. 2) were annotated as members of existing PBT gene sets: CATs, tGRITs, oGRITs, IRITs, NIRITs, GSTs, CISTS, RTs, and Slcs. We therefore looked at gene set scores in the LD, cluster 2, and cluster 3 kidneys (FIG. 4, 5). PBT scores are defined as fold-change relative to nephrectomy controls, averaged over all probesets within each PBT. Mean PBT gene set scores for Clusters 1, 2, and 3 were stratified according to the presence or absence of DGF. Only those genes passing the non-specific (IQR) filtering step were used to calculate the scores. Cluster 3 (“high risk”) was subdivided into samples with and without DGF. A continuum of severity of renal injury appeared to extend from LD to ‘Low Risk’ to ‘High Risk’ kidneys. Within the ‘High Risk’ group, those with DGF had significantly increased transcript scores for tGRITs, mCATs, IRITs, and NIRITs, compared to those with IGF (FIG. 4), reflecting greater injury, gamma interferon effects, and T cell burden.

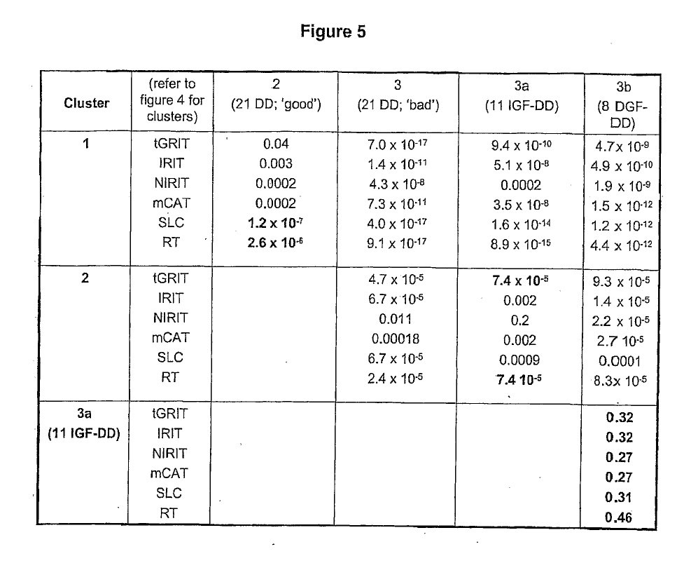

FIG. 5 shows P-values from Bayesian t-tests comparing inter-cluster PBT scores. The p-values were corrected using Benjamini and Hochberg's false discovery rate method. Again, Cluster 3 (“high-risk”) was subdivided into samples with and without DGF.

Studies were then conducted to determine whether these gene sets predicted early function in ROC analysis. FIG. 7 shows ROC curves for individual PBT scores (RTs, tGRITs, mCATs) or PC1 scores in predicting DGF status in the 42 DD kidneys. The PC1 scores were based on PBTs and on genes that were IQR filtered. Thus, the gene sets have predictive value for early function in human kidney transplants.

### Example 15

### Transcript Changes Correlate with Kidney Function in Human Kidney Transplant Biopsies and with Recovery of Function

The gene sets were assessed for their correlations with function, with change in function, and with recovery 3 months after the biopsy. The analysis includes 136 biopsies for cause. The values shown are the correlation coefficients of the log 2 of the geomeans for each gene set shown, with the statistical significance of the correlation indicted as dark green (p<0.01) or light green p<0.05).

The results indicate the gene sets correlate with function (GFR) at the time of biopsy and 3 months after the biopsy (FIG. 8). Moreover, certain gene sets correlated with the degree of loss of function/GFR before the biopsy (FIG. 9) and recovery of function/GFR after the biopsy (FIG. 9). The best correlations were with the IRITs, especially the IRITsD3 and IRITsD5 (FIGS. 10 and 11).

### Example 16

### Assessing Tissue Rejection

Epithelial deterioration is a feature of kidney allograft rejection, including invasion by inflammatory cells (tubulitis) and late tubular atrophy. Epithelial changes in CBA mouse kidneys transplanted into B6 or BALB/c wild-type (WT) or CD103 deficient (CD103^{-/-}) recipients were studied. Histology was dominated by early interstitial mononuclear infiltration from day 3 and slower evolution of tubulitis after day 7. Epithelial deterioration and tubulitis were associated with increased CD103⁺ T cells, but kidney allografts rejecting in CD103^{-/-} hosts manifested tubulitis indistinguishable from WT hosts. By microarray analysis, reduced expression of renal epithelial transporter transcripts was observed as early as day 3, indicating that renal epithelium in kidney allograft rejection deteriorates before the onset of tubulitis. Expression decreased progressively through day 42. By day 21, E-cadherin and Ksp-cadherin protein expression was reduced and redistributed. Allografts rejecting in hosts deficient in CD103, perforin, granzyme A and B, or mature B cells exhibited the same epithelial deterioration as WT hosts. These results demonstrate that the alloimmune response induces early molecular changes in the tubular epithelium that precede morphologic changes, and late changes with tubulitis and loss of cadherins, independent of CD103, cytotoxic molecules, or antibody acting on the graft. These results also demonstrate that tubulitis is a late manifestation of loss of epithelial integrity in rejection and may be a consequence rather than a cause of epithelial deterioration.

### Methods and Materials

### Mice

CD103 (Itgae) knockout mice (Schon et al., J. Immunol., 1999; 162(11):6641-6649) (CD103^{-/-}) received from Dr. C. M. Parker were bred at the University of Maryland. Other mouse strains were from Jackson Laboratory (Bar Harbor, Me.).

To confirm that the CD103^{-/-} mice were homozygous, PCR on genomic DNA was performed using primer sequences flanking the inserted neomycin resistance gene as described elsewhere (Schon et al., J. Immunol., 162(11):6641-6649 (1999)).

### Transplants

Non-life-supporting renal transplants were performed as described elsewhere (Halloran et al., J. Immunol., 166:7072-7081 (2001)) using wild-type CBA/J (H-2K^{k}) mice (CBA) as donors and wild-type C57B1/6J (H-2K^{b}) (B6), BALB/c (H-2D, I-A^{d}) (Jabs et al., Am. J. Transplant, 2003; 3(12):1501-1509) or CD103^{-/-} (on a BALB/c background) as recipients. Hosts did not receive immunosuppression. Contralateral host kidney and naïve CBA kidney served as controls. Kidneys were harvested on days 3, 4, 5, 7, 14, 21, and 42 post transplant, snap-frozen in liquid nitrogen, and stored at −70° C. until further analysis.

### Ischemic Acute Tubular Necrosis

Ischemic injury to the kidney was produced by clamping the left renal pedicle for 60 minutes in three wild-type C57B1/6J mice. Mice were sacrificed at day 7, and kidneys were harvested as described elsewhere (Goes et al., Transplantation, 59:565-572 (1995)), snap-frozen in liquid nitrogen, and stored at −70° C. until further analysis.

### Antibodies

Antibodies were obtained as follows. Rat monoclonal antibody to E-cadherin was obtained from Calbiochem-Novabiochem Corporation (San-Diego Calif.); mouse monoclonal antibody to Ksp-cadherin was obtained from Zymed Laboratories Inc. (San Francisco, Calif.); HRP-conjugated goat affinity purified F(ab′)2 to rat IgG was obtained from ICN Pharmaceuticals, Inc. (Aurora, Ohio); HRP-conjugated rabbit anti-rat and HRP-conjugated goat anti-mouse antibody were obtained from Jackson Immunoresearch Laboratories Inc. (West Grove, Pa.); anti-mouse FcγRIII/II antibody was obtained from BD Pharmingen (Mississauga, ON, Canada); anti-CD3c and anti-CD103 were obtained from eBioscience (San Diego, Calif.); and anti-CD4 and anti-CD8 were obtained from BD Pharmingen.

### Histology and Electron Microscopy

For each sample (normal kidneys, isografts, allografts, contralateral host kidneys, and ATN kidneys), frozen tissue sections (2 μm) were stained with periodic acid-Schiff (PAS) and subjected to histologic analysis as described elsewhere (Jabs et al., Am. J. Transplant., 3(12):1501-1509 (2003)). Electron microscopy was performed on glutaraldehyde-fixed tissue.

### Immunohistochemistry

Cryostat sections (4 μm) were incubated with primary antibodies to E-cadherin or Ksp-cadherin or isotype IgG as control (10 μg/mL; 90 minutes at room temperature), followed by secondary peroxidase-conjugated antibodies (1 mg/mL; 1:25 dilution; 90 minutes at room temperature). Slides were developed with diaminobenzidine tetrahydrochloride and hydrogen peroxide, and counterstained with hematoxylin. Isotype controls exhibited no immunostaining.

### Flow Cytometry

Kidney was minced, placed in 10 mL of PBS containing 2% BSA and 2 mg/mL collagenase (Sigma-Aldrich), and incubated (37° C. for 1 hour) with occasional pressing through a syringe plunger. Cells were strained, washed, and resuspended in PBS containing 0.5% FCS. Prior to flow cytometry, Fc receptors were blocked with anti-mouse FcγRIII/II antibody, and 1×10⁶ cells were stained using anti-CD3ε, anti-CD103, anti-CD4, and anti-CD8 antibodies (diluted in 0.5% FCS/PBS).

### Real-Time RT-PCR

Expression of CD103, E-cadherin, and Ksp-cadherin was assessed by TaqMan real-time RT-PCR. Total kidney RNA was extracted using CsCl density gradient. Two micrograms of RNA were transcribed using M-MLV reverse transcriptase and random primers. For laser capture microdissection (LCM), frozen sections (8 μm) were stained with the HistoGene LCM Frozen Section Staining kit (Arcturus, Mountain View, Calif.). Tubules and interstitial material were captured from day 21 transplants with the LCM instrument (Arcturus, Mountain View, Calif.), and total cellular RNA was extracted from 150 tubules and interstitial areas using the PicoPure RNA isolation kit (Arcturus). Purified RNA was reverse transcribed and amplified using the TaqMan One-Step RT-PCR kit (Applied Biosystems, Foster City, Calif.) in a multiplex reaction for 48 cycles. TaqMan probe/primer combinations were obtained as assay on demand (Applied Biosystems) (Ksp-Cadherin) or designed using Primer Express software version 1.5 (PE Applied Biosystems) (CD103: forward: 5′-CAGGAGACGCCGGACAGT-3′, SEQ ID NO:1; reverse: 5′-CAGGGCAAAGTTGCACTCAA-3′, SEQ ID NO:2; probe: 5′-AGG-AAGATGGCACTGAGATCGCTATTGTCC-3′ SEQ ID NO:3; E-Cadherin: forward: 5′-CTGCCATCCTCGGAATCCTT-3′, SEQ ID NO:4; reverse: 5′-TGGCTCAAATCAA-AGTCCTGGT-3′, SEQ ID NO:5; probe: 5′-AGGGATCCTCGCCCTGCTGATTCTGA-TC-3′, SEQ ID NO:6). Gene expression was quantified with the ABI prism 7700 Sequence Detection System (Applied Biosystems) as described elsewhere (Takeuchi et al., J. Am. Soc. Nephrol., 2003; 14(11):2823-2832). Data were normalized to HPRT mRNA, and expressed relative to the expression in control (CBA) kidneys.

### Microarrays

Microarray analysis was performed on normal kidneys (NCBA), CBA into B6 wildtype allografts at days 3, 4, 5, 7, 14, 21, and 42 posttransplant (WTD3 to WTD42), CBA into Balb/c. wildtype and CBA into Balb/c.CD103^{-/-} allografts at day 21 (CD103^{-/-} D21), CBA into CBA isografts at days 5, 7, and 21 posttransplant (Iso D7 to Iso D21), contralateral B6 host kidneys at day 5, ATN kidneys at day 7, as well as on a mixed lymphocyte culture (MLR) and cultured effector lymphocytes (CTL) (Einecke et al., Am. J. Transplant., 5(4):651-661 (2005)).

RNA extraction, dsDNA and cRNA synthesis, hybridization to MOE430A or MOE430 2.0 oligonucleotide arrays (GeneChip, Affymetrix), washing and staining were carried out according to the Affymetrix Technical Manual (See, e.g., Affymetrix Technical Manual, 2003 version downloaded from Affymetrix's website) and as described elsewhere (Einecke et al., Am. J. Transplant., 5(4):651-661 (2005)). Equal amounts of RNA from 3 mice (20-25 μg each) were pooled for each array. For NCBA, allografts, isografts, and contralateral host kidneys, two replicate chips were analyzed at each time point (two independent pools of 3 mice).

Data were normalized and analyzed with Microarray Suite Expression Analysis 5.0 software (Affymetrix) and GeneSpring™ software (Version 6.1, Silicon Genetics, CA, USA) as described elsewhere (Einecke et al., Am. J. Transplant., 5(4):651-661 (2005)).

Expression of epithelial transporter transcripts as a reflection of epithelial function (glucose transporters, amino acid transporters, and aquaporins) was analyzed. To identify those that are specific for kidney epithelium, the transporters that were present in normal kidney and had 5-fold lower expression or were absent in MLR or CTL were selected. For those transcripts that were represented by more than one probeset on the array, the probeset with annotation “_at” was selected.

### Western Blots

About 40 mg of kidney was homogenized in buffer (0.1% Nonidet P-40, 0.05% sodium deoxycholate, 0.01% SDS, 150 mM NaCl, 40 mM Tris-HCl pH 7.6, 10 mM 2-mercaptoethanol), treated with 60 μg/ml of PMSF (30 minutes on ice) then centrifuged (18,000×g; 15 minutes). 150 μg of protein (determined by Bradford reagent, Sigma-Aldrich) were run on 7.5% SDS-PAGE mini-gels (Bio-Rad, Mississauga, ON, Canada) and wet-transferred to Hybond C+ membranes (Amersham Biosciences, Baie d'Urfe, QB, Canada). Quality of transfer and evenness of loading was confirmed with Ponceau S (Sigma-Aldrich). Samples were destained in TBST (140 mM NaCl, 40 mM Tris-HCl pH 7.6, 0.1% Tween 20) and blocked with 5% milk-TBST. To preserve E-cadherin epitopes, all solutions contained 10 mM CaCl₂. Blots were incubated with primary antibodies in 5% albumin-TBST overnight (3 μg/mL, 4° C.), washed with TBST, and incubated with secondary antibodies (1:5000 in 1% milk/TBST; 1 hour at room temperature). After washing, immune complexes were detected with the ECL reagent (Amersham Biosciences) using Fuji Super RX films. Developed films were scanned using GS-800 densitometer and quantified using Quantity One software (Bio-Rad).

### Results

### Development of Interstitial Infiltrate and Tubulitis

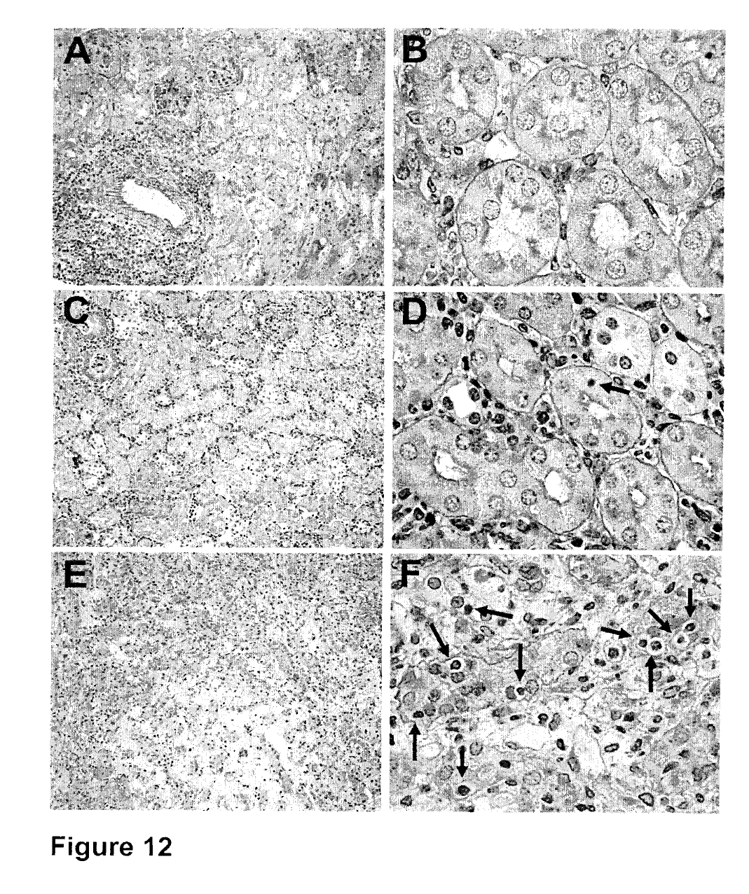

As described elsewhere (Jabs et al., Am. J. Transplant., 3(12):1501-1509 (2003); and Einecke et al., Am. J. Transplant., 5(4):651-661 (2005)), allografts exhibited focal periarterial mononuclear infiltrate at day 3 and 4 and interstitial mononuclear infiltration by day 5 (FIG. 12A), which increased at day 7 and persisted through day 42. Tubulitis was absent at day 3, 4, and 5 (FIG. 12A) and minimal at day 7, with preserved tubule structure. By day 14, 21, and 42, tubulitis was severe with distortion and shrinkage of tubule cross-sections (FIG. 12B), accompanied by endothelial arteritis. The late grafts at days 14, 21, and 42 exhibited severe tubular damage with patchy cortical necrosis (30% of the cortex by day 42). By immunostaining, the infiltrate in kidney allografts at days 5, 7, and 21 contained 40-60% CD3⁺ T cells. At day 21, T cells were present in the interstitium and tubules, with CD3⁺CD8⁺ cells exceeding CD3⁺CD4⁺ cells by 8 to 1 (34±4 versus 4±2 cells per 10 HPF, n=9). The infiltrate was 35-50% CD68⁺ (macrophages), with late appearance of 5% CD19⁺ B cells at day 21. Detailed histology results were summarized (Table 23). Host kidneys and isografts at days 5, 7, and 21 appeared normal with no inflammation or tubulitis.

A set of cytotoxic T lymphocyte-associated transcripts (CATs) was detectable by day 3 and highly expressed by day 5 in rejecting kidneys, with a median signal to 14 percent of that in cultured effector CTL, compared to 4% in isografts and normal kidneys (Einecke et al., Am. J. Transplant., 5(4):651-661 (2005)). Expression of CATs was established before diagnostic lesions and remained remarkably consistent through day 42 despite massive alterations in the pathology, and probably reflects T cells recruited to the graft.

### Expression of CD103 in Rejecting Allografts

T cells expressing integrin αEβ7 (CD103) are associated with tubulitis lesions, and αEβ7 has been implicated in the pathogenesis of tubulitis. The possibility that CD103⁺ effector T cells engage and alter tubular epithelium via CD103/E-cadherin interactions to mediate tubulitis, loss of cadherins, and deterioration of epithelial cell function was examined.

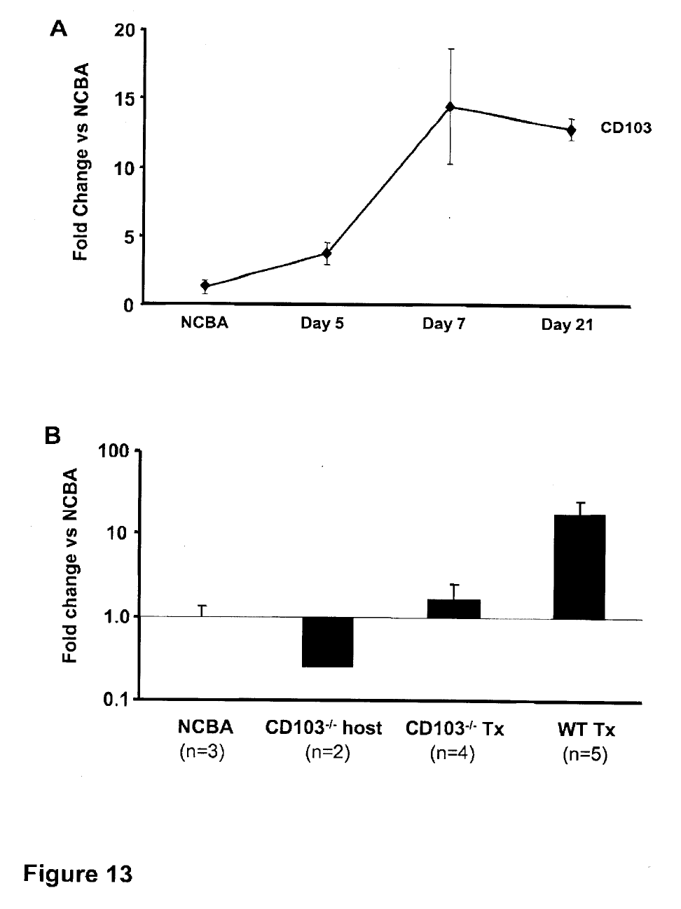

Flow cytometric analysis of lymphocytes isolated from rejecting kidneys at day 21 post transplant revealed that 32.3±13.7 percent of CD4⁺ cells and 22.0±9.2 percent of CD8⁺ cells expressed CD103 (n=3), confirming that CD103⁺ T cells are present in the rejecting graft (Hadley et al., Transplant, 1999; 67(11):1418-1425). By RT-PCR analysis, CD103 mRNA increased 4-fold at day 5 and 14-fold at day 7 post transplant (FIG. 13A), and remained 12-fold elevated at day 21. Because the CD103 antibody was unreliable for localizing CD103⁺ cells, the presence of CD103 mRNA in the epithelium was confirmed using laser capture microdissection. In kidneys with established tubulitis (day 21), CD103 mRNA was present in tubules, and was at least as abundant (91-fold) as in the interstitial infiltrate (42-fold) compared to normal kidney.

Absence of CD103 does not Prevent Tubulitis and Epithelial Deterioration

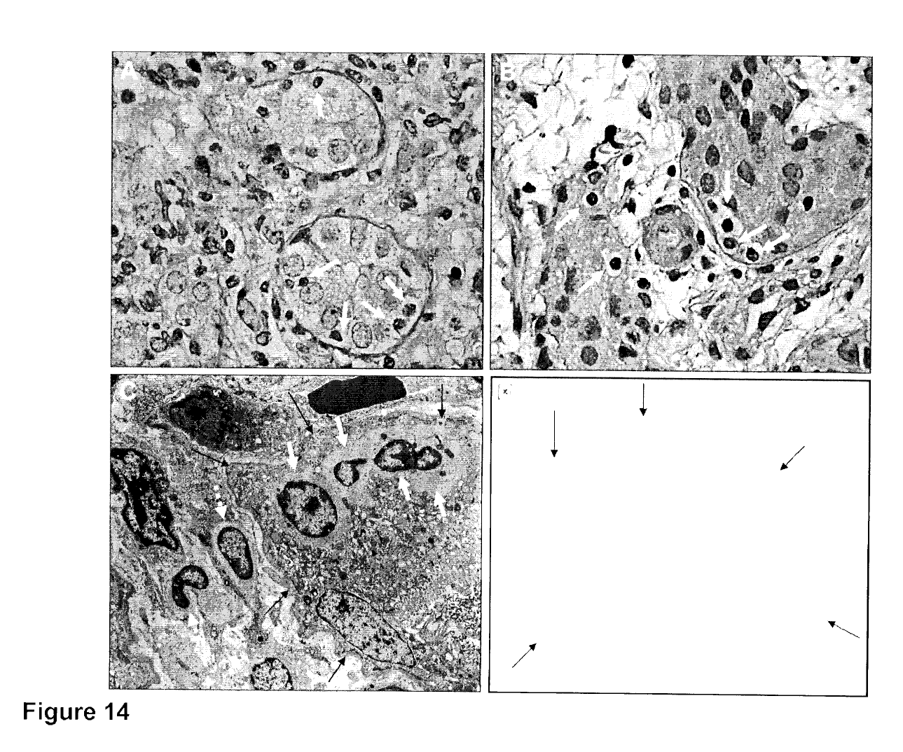

Renal allografts transplanted into CD103^{-/-} hosts at day 21 post transplant were 30 studied. As expected, CD103 RNA was absent in contralateral host kidneys and allografts in CD103^{-/-} hosts (FIG. 13B). The histologic findings in allografts rejecting in CD103^{-/-} hosts were indistinguishable from those in BALB/c wild-type hosts (FIGS. 14A and 14B), with edema, distortion of tubules, and florid tubulitis. Electron micrographs of the tubulitis lesions in kidneys rejecting in CD103^{-/-} hosts versus controls revealed no differences. In both, the intra-epithelial inflammatory cells were observed tightly applied to the basement membranes (FIGS. 14C and 14D). Semi-quantitative assessment of histologic lesions in CD103^{-/-} hosts revealed no differences (Table 24). Expression of CATs correlated highly with that in kidneys rejecting in wild-type hosts (r=0.94), confirming a similar T cell burden in the graft.

### Expression of Transporters in Rejecting Kidney as Indicators of Epithelial Deterioration

To examine epithelial function and integrity in allografts, gene expression levels for selected transporters (glucose transporters, amino acid transporters, and aquaporins) were analyzed. Transcript levels were determined by analysis of Affymetrix Genechip MOE430A or MOE430 2.0 and are represented as signal strength for normal kidney (NCBA) and fold change compared to NCBA for wild-type allografts at days 3-42 post transplant, isografts, contralateral host kidneys, ATN kidneys, and cultured lymphocytes (MLR and CTL).

### 1. Glucose Transporter Transcripts

Eight facilitated glucose transporters were represented on the chip (Table 25), six of them present in NCBA (Slc2a1, Slc2a2, Slc2a4, Slc2a5, Slc2a8, Slc2a9). Slc2a1, Slc2a8, and Slc2a9 were excluded because they were highly expressed in lymphocytes and thus not specific for epithelial cells. Slc2a2, Slc2a4, and Slc2a5 had low expression in CTL. These transcripts decreased in rejecting transplants at day 5 by at least 60 percent and continued to decrease during the course of rejection. Their expression in isografts decreased but to a lesser degree than in allografts (14%, 24%, and 33%, respectively) and was stable or recovered after day 5.

Three glucose transporters in the Na⁺-Glucose-Cotransporter family (Slc5a1, Slc5a2, and Slc5a10) actively transport glucose across the apical brush border of kidney epithelial cells. All were present in NCBA with little or no expression in lymphocytes. Slc5a2 (S1 part of proximal tubulus) and Slc5a10 decreased by 60 percent and 78 percent at day 5 and continued to decrease during the course of rejection, while Slc5a1 (S3 part of proximal tubule) decreased only after day 21. The decrease in isografts was less and was stable or improving at days 7 and 21.

Thus, transcripts for the glucose transporters in the proximal convoluted tubule (Slc2a2 and Slc5a2), where the majority of glucose re-absorption occurs, were decreased early in the course of rejection. Two transporters in the S3 segment of the proximal tubule were either not affected (Slc2a1) or decreased late (Slc5a1).

### 2. Amino Acid Transporter Transcripts

Of 29 amino acid transporters represented on the array, ten were present in NCBA with low expression in CTL (Table 26). These include neutral amino acid transporters (Slc7a7, Slc7a8, Slc7a9, Slc7a10, Slc7a12, Slc7a13, and Slc1a4), Slc3a1 (a cystine, dibasic, and neutral amino acid transport), Slc1a1 (a high affinity glutamate transport), and a neurotransmitter transporter (Slc6a13). Expression of transcripts for all transporters except Slc1a4 was decreased early in rejecting transplants (mean expression at day 5: 45 percent±17 percent of expression in NCBA) and continued to decrease over time (mean expression at day 42: 22 percent±8 percent of expression in NCBA). Slc1a4 increased early in rejection (2.3 fold) and decreased after day 21. The change in transcript expression was less in isografts (mean expression at day 5: 80 percent±44 percent of NCBA) and recovered by day 21 (100 percent±51 percent of NCBA).

### 3. Aquaporin Transcripts

Aquaporins 1, 2, 3, and 4 were present and highly expressed in normal kidney (Table 27). By day 5, mean expression of these aquaporins decreased to 0.45 percent±11 percent of expression in NCBA and continued to decrease throughout the course of rejection to 24±8 percent by day 42. Aquaporins 1, 2, and 3 were very stable in isografts, contralateral host kidneys, and ATN kidneys. Expression of aquaporin 4 was decreased in Iso D7, in ATN kidney, and in contralateral host kidneys, although to a lesser extent than in rejecting kidneys. Aquaporins 5, 7, and 9 were absent in NCBA and throughout the rejection process.

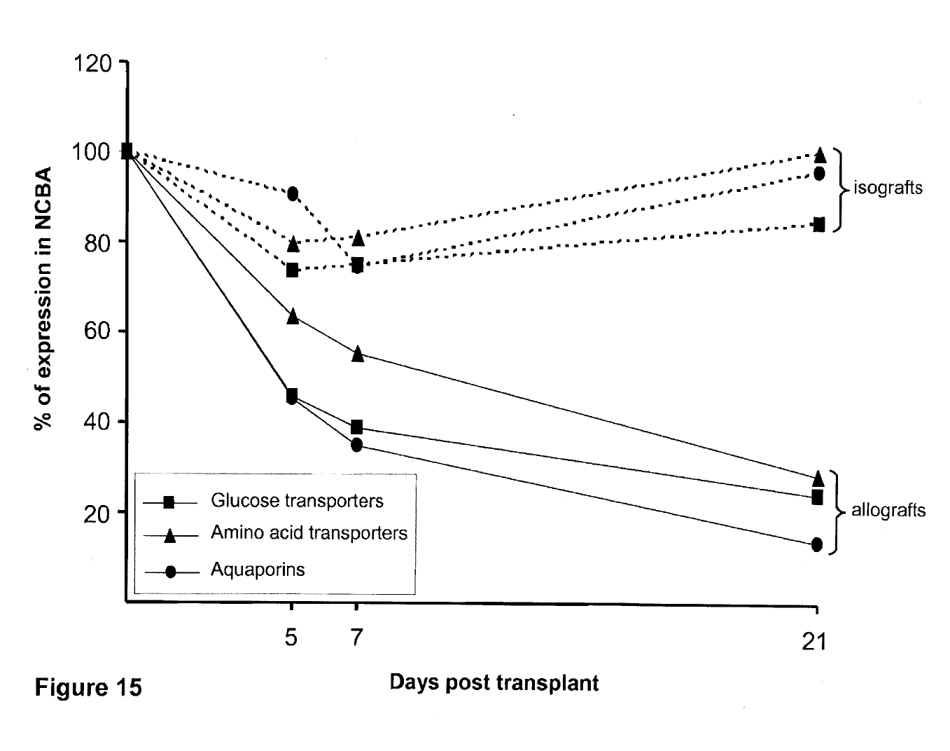

The results for glucose and amino acid transporters and for aquaporins are summarized in FIG. 15, illustrating how many epithelial transport transcripts in rejecting kidneys are depressed at day 5 by a mechanism requiring the allo-response, but before the development of significant tubulitis.

In allografts rejecting in CD103^{-/-} recipients, a decreased expression of glucose transporters, amino acid transporters and aquaporins similar to that in wild-type hosts was observed (Table 28), with a correlation coefficient r=0.84.

### Cadherins in Rejecting Kidneys

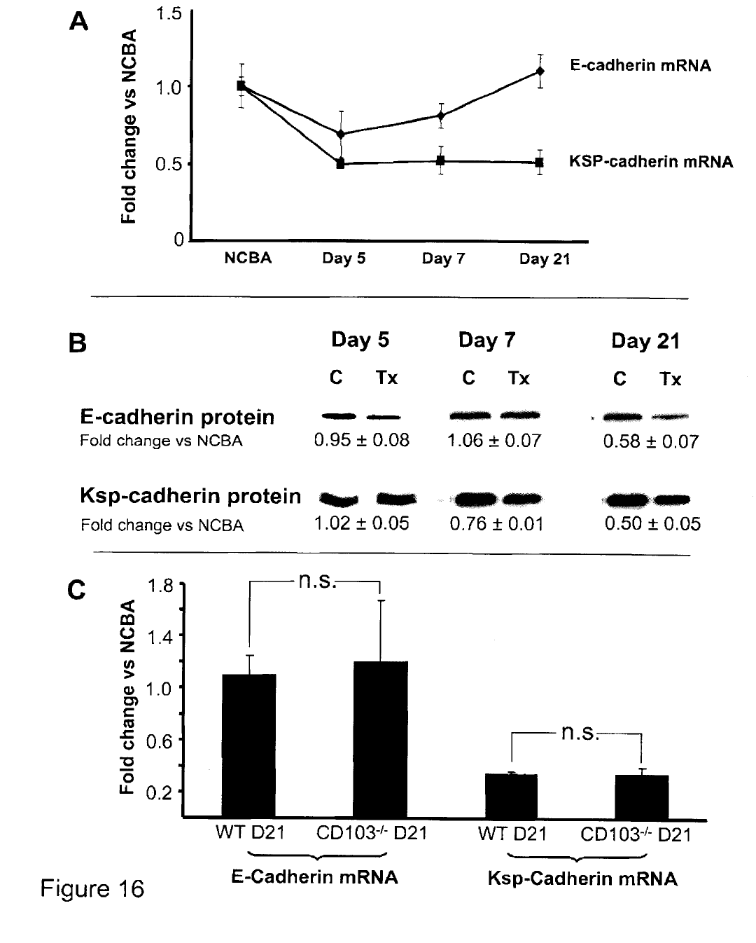

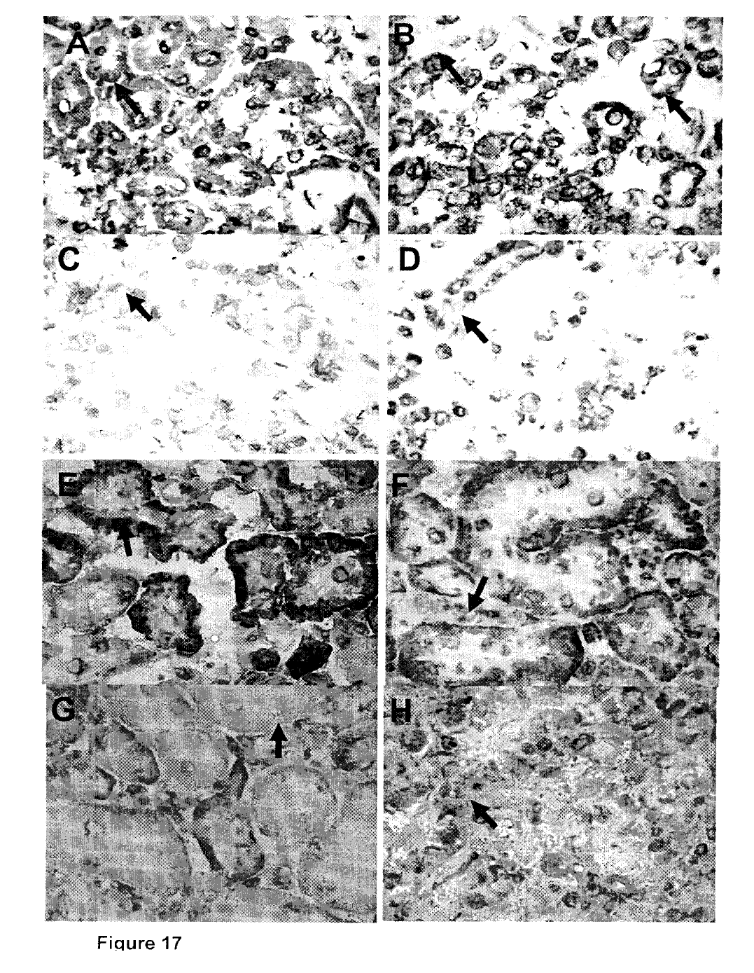

E-cadherin mRNA levels fell only transiently in rejecting kidney at day 5 (FIG. 16A). Western blot analysis confirmed this finding, revealing that E-cadherin protein decreased in rejecting kidney by 40 percent at day 21 compared to the contralateral control kidney (FIG. 16B), suggesting that post-transcriptional mechanisms contribute to the reduced E-cadherin staining. By immunostaining, E-cadherin was expressed on the basolateral membrane of tubular epithelial cells in control kidney (CBA) and in the contralateral host kidney at day 7 (FIG. 17A) and day 21. All tubules were positive for E-cadherin in the basolateral membrane, although the intensity was highly variable among tubules. In rejecting allografts, staining intensity was unchanged at day 7 post transplant (FIG. 17B), but by day 21 E-cadherin staining was both severely decreased and redistributed, with loss of polarity manifested by staining of the luminal membrane and loss of basolateral staining in some tubules (FIG. 17C).

Ksp-cadherin mRNA decreased by 50 percent at day 5 post transplant and remained depressed through day 21 (FIG. 16A). Western blots revealed decreased protein level at day 7 (25 percent) and 21 (50 percent) post allograft (FIG. 16B). Staining for Ksp-cadherin in normal control kidneys was similar to that for E-cadherin (FIG. 17E). In rejecting kidney, Ksp-cadherin staining intensity was lower at day 7 (FIG. 17F) and greatly diminished and redistributed at day 21 (FIG. 17G), similar to changes in E-cadherin.

Comparison of day 21 CBA allografts rejecting either in BALB/c or CD103^{-/-} hosts revealed that the decrease in Ksp-Cadherin mRNA and the persistence of E-Cadherin mRNA was similar in both groups (FIG. 16C). E-Cadherin and Ksp-Cadherin staining was decreased in the allografts rejecting in CD103^{-/-} hosts at day 21, similar to the findings in wild-type hosts (FIG. 17D and 17H, respectively).

### Epithelial Deterioration is T-Cell Mediated but not Dependent on Cytotoxicity

A decrease in expression of epithelial transporters and cadherins was observed in kidneys rejecting in hosts lacking perforin, granzyme A and B, or mature B-cells, similar to those in wild-type hosts (Table 29).

Lengthy table referenced here
US20090176656A1-20090709-T00001
Please refer to the end of the specification for access instructions.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

LENGTHY TABLES
The patent application contains a lengthy table section. A copy of the table is available in electronic form from the USPTO web site (http://seqdata.uspto.gov/?pageRequest=docDetail&DocID=US20090176656A1). An electronic copy of the table will also be available from the USPTO upon request and payment of the fee set forth in 37 CFR 1.19(b)(3).

1-101. (canceled)

102. An ex vivo method for detecting tissue injury, wherein said method comprises determining whether or not a tissue contains cells having a solute carrier profile, or
wherein said method comprises determining whether or not a tissue contains cells having an injury and repair profile, or
wherein said method comprises determining whether or not a tissue contains cells having a not-in-isografts injury and repair profile, or
wherein said method comprises determining whether or not a tissue contains cells having a gamma interferon (IFN-γ) suppressed profile, or
wherein said method comprises determining whether or not a tissue contains cells having a class I suppressed profile, or
wherein said method comprises determining whether or not a tissue contains cells having a renal transcript (RT) profile, or
wherein said method comprises determining whether or not a tissue contains cells having an injury and repair correlated profile or an Slc correlated profile, or
comprising determining whether or not a tissue contains cells having increased activity of biochemical pathways that correlate with an injury and repair profile, with an Slc profile, with a not-in-isografts injury and repair profile, with a gamma interferon suppressed profile, with a class I suppressed profile, or with an RT profile, wherein the presence of said cells indicates that said tissue is injured.

103. The method of claim 102, wherein said tissue is transplanted and the presence of said cells indicates that said tissue is not likely to recover from injury, or
wherein the presence of said cells indicates that said tissue is at risk for delayed graft function (DGF).

104. The method of claim 102, wherein said mammal is a human.

105. The method of claim 102, wherein said tissue is from a biopsy, or is kidney tissue, or is to be transplanted into a recipient, or has been transplanted into a recipient.

106. The method of claim 102, wherein said determining step comprises using PCR, a nucleic acid array, immunohistochemistry, or an array for detecting polypeptides.

107. The method of claim 102, wherein said biochemical pathways correlate with an injury and repair profile, or correlate with an Slc profile.

108. An apparatus for determining whether a tissue is injured, said apparatus comprising:
one or more collectors for obtaining signals representative of the presence of one or more nucleic acids listed in Tables 1-14, 19, and 20 in a sample from said tissue; and
a processor for analyzing said signals and determining whether said tissue is injured.

109. The apparatus of claim 108, wherein said one or more collectors are configured to obtain further signals representative of the presence of said one or more nucleic acids in a control sample.

110. A nucleic acid array comprising at least 20 nucleic acid molecules, wherein each of said at least 20 nucleic acid molecules has a different nucleic acid sequence, and wherein at least 50 percent of the nucleic acid molecules of said array comprise a sequence from nucleic acid selected from the group consisting of the nucleic acids listed in Tables 1-14, 19, and 20.

111. The array of claim 110, wherein said array comprises at least 50 or 100 nucleic acid molecules, wherein each of said at least 50 or 100 nucleic acid molecules has a different nucleic acid sequence.

112. The array of claim 110, wherein each of said nucleic acid molecules that comprise a sequence from nucleic acid selected from said group comprises no more than three mismatches.

113. The array of claim 110, wherein at least 75 or 95 percent of the nucleic acid molecules of said array comprise a sequence from nucleic acid selected from said group.

114. The array of claim 110, wherein said array comprises glass.

115. The array of claim 110, wherein said at least 20 nucleic acid molecules comprise a sequence present in a human.

116. A computer-readable storage medium having instructions stored thereon for causing a programmable processor to determine whether one or more nucleic acids listed in Tables 5-14, and the third column of Table 20 are present in a tissue sample at elevated levels, or is expressed at a greater level in said tissue sample than in a control tissue sample.

117. A computer-readable storage medium having instructions stored thereon for causing a programmable processor to determine whether one or more nucleic acids listed in Tables 1-4 and the third column of Table 19 are present in a tissue sample at decreased levels, or is expressed at a lower level in said tissue sample than in a control tissue sample.
